SEQUENCE LISTING (41 sequences)

SEQ ID NO: 1 - protein, 736 aa, Artificial Sequence
  Synthesized
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 2 - protein, 735 aa, Artificial Sequence [sequence omitted]

SEQ ID NO: 3 - protein, 736 aa, Artificial Sequence
  Synthesized
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 4 - protein, 734 aa, Artificial Sequence
  Synthesized
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 5 - protein, 724 aa, Artificial Sequence
  Synthesized
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 6 - protein, 736 aa, Artificial Sequence
  Synthesized
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 7 - protein, 737 aa, Artificial Sequence
  Synthesized
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 8 - protein, 738 aa, Artificial Sequence
  Synthesized
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 9 - protein, 736 aa, Artificial Sequence
  Synthesized
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 10 - protein, 738 aa, Artificial Sequence
  Synthesized
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGESESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQANTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 11 - protein, 733 aa, Artificial Sequence
  Synthesized
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPARKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 12 - protein, 736 aa, Artificial Sequence
  Synthesized
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMANQARNWVPGPCYRQQRVSTTTNQNNNSNFAWTGAAKFKLNGRDSLMNPGVAMASHKDDDDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 13 - protein, 738 aa, Artificial Sequence
  Synthesized
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 14 - protein, 735 aa, Artificial Sequence
  Synthesized
MAADGYLPDWLEDTLSEGIRQWWKLKPGPPPPKPAERHQDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGHQPARKRLNFGQTGDADSVPDPQPLGQPPAAPTSLGSTTMATGSGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLNRTQSNSGTLQQSRLLFSQAGPTSMSLQAKNWLPGPCYRQQRLSKQANDNNNSNFPWTAATKYRLNGRDSLVNPGPAMASHKDDEEKFFPMHGTLIFGKQGTNANDADLEHVMITDEEEIRTTNPVATEQYGNVSNNLQNSNTGPTTENVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 15 - protein, 735 aa, Artificial Sequence
  Synthesized
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGQQPARKRLNFGQTGDADSVPDPQPLGQPPAAPSGLGTNTMASGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGGNTQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 16 - protein, 738 aa, Artificial Sequence
  Synthesized
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 17 - protein, 738 aa, Artificial Sequence
  Synthesized
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKTGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 18 - protein, 736 aa, Artificial Sequence
  Synthesized
MAADGYLPDWLEDNLSEGIREWWDLKPGAPKPKANQQKQDDGRGLVLPGYKYLGPFNGLDKGEPVNAADAAALEHDKAYDQQLEAGDNPYLRYNHADAEFQERLQEDTSFGGNLGRAVFQAKKRVLEPLGLVEEGAKTAPGKKRPVEQSPQEPDSSSGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPVTGSCFWQQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 19 - protein, 738 aa, Artificial Sequence
  Synthesized
MAADGYLPDWLEDNLSEGIREWWALKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDGVITTSTRTWALPTYNNHLYKQISNGTSGGSTNDNTYFGYSTPWGYFDFNRFHCHFSPRDWQRLINNNWGFRPKSLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNDNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 20 - protein, 736 aa, Artificial Sequence
  Synthesized
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL

SEQ ID NO: 21 - protein, 737 aa, Artificial Sequence
  Synthesized
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTTNTQTLGFSQGGPNTMANQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 22 - protein, 735 aa, Artificial Sequence
  Synthesized
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQQQNTAPATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 23 - protein, 735 aa, Artificial Sequence
  Synthesized
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQEGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 24 - protein, 735 aa, Artificial Sequence
  Synthesized
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSVAGPSNMAVQGRNWLPGPCYRQQRVSKTSADNNNSEFAWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 25 - protein, 735 aa, Artificial Sequence
  Synthesized
MAADGYLPDWLEDNLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKTGQQPAKKRLNFGQTGDSESVPDPQPLGEPPAAPSGLGTNTMATGSGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEEFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHHSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 26 - protein, 735 aa, Artificial Sequence
  Synthesized
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKTGQQPAKKRLNFGQTGDSESVPDPQPLGEPPAAPSGLGTNTMATGSGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWAGATKYHLNGRDSLVNPGPAMASHKDDEEKFFPQSGVLIFGKQGSEKTNVDIEKVMITDEEEIRTTNPVATEQYGSVSTNLQRGNRQAATADVDTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 27 - protein, 743 aa, Artificial Sequence
  Synthesized
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 28 - protein, 743 aa, Artificial Sequence
  Synthesized
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 29 - protein, 743 aa, Artificial Sequence
  Synthesized
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQQAVRTSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 30 - protein, 5 aa, artificial sequence
  peptide linker
GGGGG

SEQ ID NO: 31 - protein, 5 aa, artificial sequence
  peptide linker
GGSGG

SEQ ID NO: 32 - protein, 5 aa, artificial sequence
  peptide linker
GSGGG

SEQ ID NO: 33 - protein, 736 aa, Artificial Sequence
  Synthesized
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKKGQQPARKRLNFGQTGDSESVPDPQPLGEPPAAPSGVGSNTMAAGGGAPMADNNEGADGVGNASGNWHCDSTWLGDRVITTSTRTWALPTYNNHLYKQISSQSGGSTNDNTYFGYSTPWGYFDFNRFHCHFSPRDWQRLINNNWGFRPKKLNFKLFNIQVKEVTTNDGTTTIANNLTSTVQVFTDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLSRTQTTSGTAGNRTLQFSQAGPSSMANQAKNWLPGPCYRQQRVSKTTNQNNNSNFAWTGATKYHLNGRDSLVNPGPAMATHKDDEDKFFPMSGVLIFGKQGAGNSNVDLDNVMITNEEEIKTTNPVATEEYGTVATNLQSANTAPATGTVNSQGALPGMVWQDRDVYLQGPIWAKIPHTDGHFHPSPLMGGFGLKHPPPQILIKNTPVPANPPTTFSPAKFASFITQYSTGQVSVEIEWELQKENSKRWNPEIQYTSNYNKSTNVDFAVDTNGVYSEPRPIGTRYLTRNL

SEQ ID NO: 34 - protein, 745 aa, Artificial Sequence
  Synthesized
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGETTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 35 - protein, 736 aa, Artificial Sequence
  Synthesized
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNVQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQDQSGSAQNKDLLFSRGSPAGMSVQPKNWLPGPCYRQQRVSKTKTDNNNSNFTWTGASKYNLNGRESIINPGTAMASHKDDKNKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 36 - protein, 5 aa, artificial sequence
  peptide linker
GGGSG

SEQ ID NO: 37 - protein, 5 aa, artificial sequence
  peptide linker
GGGGS

SEQ ID NO: 38 - protein, 745 aa, Artificial Sequence
  Synthesized
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNDGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLADQDYTKTARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSINVDFTVDTNGVYSEPRPIGTRYLTRNL

SEQ ID NO: 39 - protein, 737 aa, Artificial Sequence
  Synthesized
MAADGYLPDWLEDTLSEGIRQWWKLKPGPPPPKPAERHKDDSRGLVLPGYKYLGPGNGLDKGEPVNAADAAALEHDKAYDQQLKAGDNPYLRYNHADAEFQERLQEDTSFGGNLGRAVFQAKKRVLEPLGLVEEPVKTAPGKKRPVEHSPVEPDSSSGTGKAGQQPARKRLNFGQTGDTDSAADPQPLGEPPAAPSGLGTGTMAAGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTQNEGTKTIANNLTSTVQVFTDSEYQLPYVLGSAHQGCLPPFPADVFMIPQYGYLTLNNGSQAMGRSSFYCLEYFPSQMLRTGNNFQFTYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQILIKNTPVPADPPTAFNKDKLNSFITQYSTGQVSVEIEWELQKENSKRWNPEIQYTSNYYKSTSVDFAVNTEGVYSEPRPIGTRYLTRNL

SEQ ID NO: 40 - protein, 737 aa, Artificial Sequence
  Synthesized
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEHSPVEPDSSSGIGKTGQQPAKKRLNFGQTGDADSVPDPQPLGQPPAAPSGLGTNTMATGSGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTQNEGTTTIANNLTSTVQVFTDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL

SEQ ID NO: 41 - protein, 738 aa, Artificial Sequence
  Synthesized
MAADGYLPDWLEDTLSEGIRQWWKLKPGPPPPKPAERHKDDSRGLVLPGYKYLGPFNGLDKGEPVNEADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

(19) United States
(12) Patent Application Publication
(10) Pub. No.: US 2022/0354969 A1
(43) Pub. Date: Nov. 10, 2022

(54) ENHANCING VIRAL VECTOR-BASED GENE THERAPY

(71) Applicant: Oregon Health & Science University, Portland, OR (US)

(72) Inventors: Hiroyuki Nakai, West Linn, OR (US); Masahiro Horikawa, Portland, OR (US); Kei Adachi, Beaverton, OR (US)

(73) Assignee: Oregon Health & Science University, Portland, OR (US)

(21) Appl. No.: 17/740,240

(22) Filed: May 9, 2022

Related U.S. Application Data
(60) Provisional application No. 63/186,795, filed on May 10, 2021.

(51) Int. Cl. A61K 48/00

(52) U.S. Cl. CPC A61K 48/0075; C12N 2750/14143; C12N 2750/14171

(57) ABSTRACT

Disclosed herein are methods for delivering a composition comprising an adeno-associated virus (AAV) to one or more pancreatic cells in a subject. Also disclosed are adeno-associated viral vectors for use in these methods.

### FEDERALLY SPONSORED RESEARCH OR DEVELOPMENT

This invention was made with government support under DK104143 and U01 DK123608 awarded by the National Institutes of Health. The government has certain rights in the invention.

### CROSS REFERENCE TO RELATED APPLICATIONS

This application claims the benefit of U.S. application Ser. No. 63/186,785, filed May 10, 2021, which is incorporated by reference herein.

### COPYRIGHT NOTICE

© 2022 Oregon Health & Science University. A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR § 1.71(d).

### TECHNICAL FIELD

Generally, the disclosure relates to methods of delivering gene therapy. More specifically, the disclosure relates to viral vectors used in gene therapy to transduce target specific cell types in the pancreas and methods of delivering such viral vectors.

### BACKGROUND

Advances in various technologies for cell fate conversion offer an innovative approach to treat various human diseases by reprograming one cell type into another. Of relevance to the development of novel gene therapy approaches for treating many diseases such as Type 1 diabetes is the demonstration of successful transduction and reprogramming of target cells (e.g. non-beta pancreatic cells) by vector-mediated delivery of key transcription factors to the target cells. One step in translating these approaches to the clinic is establishing a clinically relevant effective and safe approach to deliver a gene therapy vector to an affected organ or tissue and showing successful gene transfer.

### SUMMARY

Methods are disclosed for delivering a composition comprising an adeno-associated virus (AAV) to one or more pancreatic cell types in a subject. These methods include: a) guiding a distal end of a catheter to a major duodenal papilla in a duodenum of the subject; b) advancing the distal end of the catheter in a retrograde direction through the pancreatic duct to a delivery point internal to the pancreas of the subject; c) delivering a therapeutically effective amount of the composition through the catheter to the delivery point; and d) allowing the composition to reside at the delivery point for a dwell time. The distal end of the catheter has a diameter selected to inhibit backflow of the AAV. The AAV includes a genome and a capsid protein, wherein the capsid protein includes the amino acid sequence of SEQ ID NO: 1, SEQ ID NO: 2, SEQ ID NO: 7, SEQ ID NO: 8, SEQ ID NO: 10, SEQ ID NO: 12, SEQ ID NO: 13, SEQ ID NO: 15, SEQ ID NO: 16, SEQ ID NO: 17, SEQ ID NO: 20, SEQ ID NO: 21, SEQ ID NO: 22, SEQ ID NO: 24, SEQ ID NO: 25, SEQ ID NO: 27, SEQ ID NO: 28, SEQ ID NO: 33, SEQ ID NO: 34, SEQ ID NO: 35, SEQ ID NO: 39, or SEQ ID NO: 41, or an amino acid sequence at least 95% identical thereto. The guiding step can include surgery or endoscopy.

In some embodiments, the pancreatic cell type is islet cells, and the capsid protein comprises the amino acid sequence of SEQ ID NO: 1, SEQ ID NO: 7, SEQ ID NO: 10, SEQ ID NO: 12, SEQ ID NO: 16, SEQ ID NO: 20, SEQ ID NO: 21, SEQ ID NO: 22, SEQ ID NO: 24, SEQ ID NO: 27, SEQ ID NO: 28, SEQ ID NO: 33, SEQ ID NO: 34, SEQ ID NO: 35, SEQ ID NO: 39, SEQ ID NO: 41 or an amino acid sequence at least 95% identical thereto.

In further embodiments, the pancreatic cell type is duct cells and the capsid protein comprises the amino acid sequence of SEQ ID NO: 1, SEQ ID NO: 2, SEQ ID NO: 7, SEQ ID NO: 8, SEQ ID NO: 10, SEQ ID NO: 12, SEQ ID NO: 13, SEQ ID NO: 15, SEQ ID NO: 16, SEQ ID NO: 17, SEQ ID NO: 20, SEQ ID NO: 21, SEQ ID NO: 24, SEQ ID NO: 25, SEQ ID NO: 28, SEQ ID NO: 33, SEQ ID NO: 35, SEQ ID NO: 39, SEQ ID NO: 41 or an amino acid sequence at least 95% identical thereto.

In more embodiments, the pancreatic cell type is acinar cells, and the capsid protein comprises the amino acid sequence of SEQ ID NO: 7, SEQ ID NO: 12, SEQ ID NO: 16, SEQ ID NO: 21, SEQ ID NO: 25, SEQ ID NO: 27, or an amino acid sequence at least 95% identical thereto.

In further embodiments, methods are disclosed for treating a pancreatic disorder in a subject, that includes performing endoscopic retrograde cholangiopancreatography to transduce pancreatic cells of the patient with an AAV comprising a genome and a capsid protein comprising the amino acid sequence of SEQ ID NO: 1, SEQ ID NO: 7, SEQ ID NO: 10, SEQ ID NO: 12, SEQ ID NO: 16, SEQ ID NO: 20, SEQ ID NO: 21, SEQ ID NO: 22, SEQ ID NO: 24, SEQ ID NO: 27, SEQ ID NO: 28, SEQ ID NO: 33, SEQ ID NO: 34, SEQ ID NO: 35, SEQ ID NO: 39, SEQ ID NO: 41, or an amino acid sequence at least 95% identical thereto, wherein the genome encodes a therapeutic protein that treats the pancreatic disorder in the subject.

Additional aspects and advantages will be apparent from the following detailed description of preferred embodiments, which proceeds with reference to the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B shows images of ampulla of Vater visualized by duodenotomy. Panel 1A is an image of the ampulla of Vater, where the orifice of the ampulla can be identified as a small wet dot in the center of the ampulla. Panel 1B is an image of a 24-gauge (G) angiocatheter (Surflo) that is inserted into the cholangio-pancreatic duct system through the orifice of the ampulla of Vater.

FIGS. 2A-2C shows images of image-guided pancreatic duct injection of agents. Panel 2A is an image of an 0.014-inch guidewire that is inserted into the main pancreatic duct, reaching the pancreatic tail. Panel 2B shows digital subtraction images of the pancreas injected with a contrast agent through a 5-French micropuncture catheter placed in the main pancreatic duct and wedged at the pancreas head and body junction. The time indicated on each image indicates the time after the initiation of injection of a small volume of the contrast agent. In this procedure, an acinar shadow can be observed. Panel 2C is a digital subtraction image taken after AAV vector injection to confirm the position and wedging condition of the catheter.

FIGS. 3A-3B shows fluorescence images of HEK293 cells exposed to AAV2-CMV-GFP vector (upper row) or AAV9-CAG-TdTomato (bottom row) (panel 3A) and a graphical representation of relative transduction efficiencies in CHO Lec2 cells across treatment with various Iohexol concentrations. One-way ANOVA shows no statistically significant difference between the groups (n.s., not significant).

FIGS. 4A-4D shows representative images of pancreatic duct injection procedure. Panel 4A is a graphical representation of AAV pancreatic duct injection. Panel 4B is an image of a laparotomy, where a duodenum wall was opened to directly visualize the ampulla of Vater. Panel 4C is a live fluoroscopy image showing insertion of a guidewire into the pancreatic duct. Panel 4D is an image of infusion of a contrast agent after AAV infusion for the confirmation of catheter position in body region.

FIG. 5 shows graphical representations of the assessment of pancreatitis in Animal 1 (left panels) and Animal 2 (right panels). Serum amylase levels (top panels) and serum lipase levels (lower panels) across various time points relative to the time of AAV vector injection are shown.

FIG. 6A is an image showing segments and segment numbers of pancreas tissue.

FIG. 6B is a color legend for the fluorescence and its associated markers shown in FIG. 6C.

FIG. 6C shows fluorescence microscopy images of head segment number 4 (left panels), body segment number 7 (center panels), and tail segment number 11 (right panels) of the pancreas in a rhesus macaque transduced with AAV9-CAG-TdTomato-injected pancreatic tissue in a rhesus macaque. Arrowheads indicate TdTomato-positive islet cells that reside in the peripheral zone in islets. Scale bars, 100 μm.

FIG. 6D is a color legend for the fluorescence and its associated markers shown in FIG. 6E.

FIG. 6E shows representative fluorescence microscopy images of the rhesus macaque pancreas tissues transduced with AAV9-CAG-TdTomato or AAVKP1-CAG-TdTomato by vector administration via the pancreatic duct.

FIG. 7 is a graphical representation of vector concentration in blood over time post-injection.

### SEQUENCE LISTING

The nucleic and amino acid sequences listed in the accompanying sequence listing are shown using standard letter abbreviations for nucleotide bases and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file, [Sequence_Listing, created on May 6, 2022, 225 KB, which is incorporated by reference herein.

SEQ ID NOs: 1-29, 33-35, and 38-41 are the amino acid sequences of capsid (VP1) proteins.

SEQ ID NOs: 30-31 and 36-37 are the amino acid sequences of linkers.

### DETAILED DESCRIPTION

Methods are disclosed for delivering a composition comprising an adeno-associated virus (AAV) to one or more pancreatic cell types in a subject. In further embodiments, methods are disclosed for treating a pancreatic disorder in a subject, that includes performing endoscopic retrograde cholangiopancreatography to transduce pancreatic cells of the patient with an AAV.

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “a vector” includes singular or plural vectors and can be considered equivalent to the phrase “at least one vector.” As used herein, the term “comprises” means “includes.” The term “about” indicates within five percent. It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting.

The term “AAV vector” as used herein means any vector that comprises or derives from components of AAV and is suitable to infect mammalian cells, including human cells, of any of a number of tissue types, such as brain, heart, lung, skeletal muscle, liver, kidney, spleen, or pancreas, whether in vitro or in vivo. The term “AAV vector” may be used to refer to an AAV type viral particle (or virion) comprising at least a nucleic acid molecule encoding a protein of interest.

The term “administration” refers to providing or giving a subject an agent by any effective route. Exemplary routes of administration include, but are not limited to, oral, injection (such as subcutaneous, intramuscular, intradermal, intraperitoneal, intravenous, and intratumoral), sublingual, rectal, transdermal, intranasal, intraductal, vaginal and inhalation routes. In some embodiments, administration is to a pancreatic duct.

An “agent” is any polypeptide, compound, small molecule, organic compound, salt, polynucleotide, or other molecule of interest. Agent can include a therapeutic agent, a diagnostic agent or a pharmaceutical agent. A “therapeutic agent” is a substance that demonstrates some therapeutic effect by restoring or maintaining health, such as by alleviating the symptoms associated with a disease or physiological disorder, or delaying (including preventing) progression or onset of a disease, such as diabetes or an insulinoma. An agent can be an AAV vector.

An “animal” is a living multi-cellular vertebrate organism, a category that includes, for example, mammals and birds. The term mammal includes both human and non-human mammals. Similarly, the term “subject” includes both human and veterinary subjects, including non-human primates.

The term “anti-diabetic lifestyle modifications” include changes to lifestyle, habits, and practices intended to alleviate the symptoms of diabetes or pre-diabetes. Obesity and sedentary lifestyle may both independently increase the risk of a subject developing type II diabetes, so anti-diabetic lifestyle modifications include those changes that will lead to a reduction in a subject's body mass index (BMI), increase physical activity, or both. Specific, non-limiting examples include the lifestyle interventions described in Diabetes Care, 22(4):623-34 at pages 626-27.

The term “conservative substitutions” of a polypeptide that involve the substitution of one or more amino acids for amino acids having similar biochemical properties that do not result in change or loss of a biological or biochemical function of the polypeptide are designated “conservative” substitutions. These conservative substitutions are likely to have minimal impact on the activity of the resultant protein. Table 1 shows amino acids that can be substituted for an original amino acid in a protein, and which are regarded as conservative substitutions.

TABLE

| Original Residue | Conservative Substitutions |
|---|---|
| Ala | ser |
| Arg | lys |
| Asn | gln; his |
| Asp | glu |
| Cys | ser |
| Gln | asn |
| Glu | asp |
| Gly | pro |
| His | asn; gln |
| Ile | leu; val |
| Leu | ile; val |
| Lys | arg; gln; glu |
| Met | leu; ile |
| Phe | met; leu; tyr |
| Ser | thr |
| Thr | ser |
| Trp | tyr |
| Tyr | trp; phe |
| Val | ile; leu |

One or more conservative changes, or up to ten conservative changes (such as two substituted amino acids, three substituted amino acids, four substituted amino acids, or five substituted amino acids, etc.) can be made in the polypeptide without changing a biochemical function of the protein, such as a capsid protein, such as the affinity for a particular cell type.

The term “detarget” refers to less than about 0.05 (5%), less than about 0.1 (10%) or less than about 0.2 (20%) of AAV vector genomes delivered to the organ of interest or AAV vector genome transcripts (transgene expression) compared to those of a benchmark (control) AAV. In some embodiments, AAV9 as the benchmark (control) AAV.

The term “diabetes” refers to a group of metabolic diseases in which a subject has high blood sugar, either because the pancreas does not produce enough insulin, or because cells do not respond to the insulin that is produced. Type 1 diabetes results from the body's failure to produce insulin. This form has also been called “insulin-dependent diabetes mellitus” (IDDM) or “juvenile diabetes”. Type 1 diabetes mellitus is characterized by loss of the insulin-producing βcells, leading to insulin deficiency. This type can be further classified as immune-mediated or idiopathic. Type 2 diabetes results from insulin resistance, a condition in which cells fail to use insulin properly, sometimes combined with an absolute insulin deficiency. This form is also called “non insulin-dependent diabetes mellitus” (NIDDM) or “adult-onset diabetes.” The defective responsiveness of body tissues to insulin is believed to involve the insulin receptor. Diabetes mellitus is characterized by recurrent or persistent hyperglycemia, and is diagnosed by demonstrating any one of:
a. Fasting plasma glucose level ≥7.0 mmol/l (126 mg/dl);
b. Plasma glucose ≥11.1 mmol/l (200 mg/dL) two hours after a 75 g oral glucose load as in a glucose tolerance test;
c. Symptoms of hyperglycemia and casual plasma glucose ≥11.1 mmol/l (200 mg/dl);
d. Glycated hemoglobin (Hb A1C) ≥6.5%

Type 1 or Type 2 diabetes can be treated using the methods disclosed here.

The term “expressed” refers to the translation of a nucleic acid into a protein. Proteins may be expressed and remain intracellular, become a component of the cell surface membrane, or be secreted into the extracellular matrix or medium.

The terms “exocrine cells” and “acinar cells” refers to the cells of the secretory tissue of the exocrine pancreas, which distributes its products, such as enzymes, via an associated duct network. The exocrine cells secrete enzymes required for digestion. The exocrine cells of the pancreas include the centroacinar cells and basophilic cells, which produce secretin and cholecystokinin.

The term “expression control sequences” refers to nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (i.e., ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

A “heterologous” sequence is a sequence that is not normally (in the wild-type sequence) found adjacent to a second sequence. In one embodiment, the sequence is from a different genetic source, such as a virus or organism, than the second sequence.

The term “islet cells” include the endocrine cells of the pancreas, which are found in the islets of Langerhans in the pancreas in vivo. A “pancreatic endocrine cell” produces one or more pancreatic hormone, such as insulin, glucagon, somatostatin, or pancreatic polypeptide. Subsets of pancreatic endocrine cells include the α (glucagon producing), β (insulin producing) δ (somatostatin producing) or PP (pancreatic polypeptide producing) cells. “Alpha (α) cells” are mature glucagon producing cells of the islets. “Beta (β) cells” are mature insulin producing cells. “Delta (δ)” cells are mature somatostatin producing cells of the islets. “PP cells” are the mature pancreatic peptide (PP) producing cells of the islets.

Additional subsets produce more than one pancreatic hormone, such as, but not limited to, a cell that produces both insulin and glucagon, or a cell that produces insulin, glucagon, and somatostatin, or a cell that produces insulin and somatostatin.

An “isolated” biological component (such as a nucleic acid, peptide or protein) has been substantially separated, produced apart from, or purified away from other biological components in the cell of the organism in which the component naturally occurs, i.e., other chromosomal and extrachromosomal DNA and RNA, and proteins. Nucleic acids, peptides and proteins which have been “isolated” thus include nucleic acids and proteins purified by standard purification methods. The term also embraces nucleic acids, peptides and proteins prepared by recombinant expression in a host cell as well as chemically synthesized nucleic acids. An isolated cell type has been substantially separated from other cell types, such as a different cell type that occurs in an organ. A purified cell or component can be at least 90%, at least 95%, at least 96%, at least 97%, at least 98%, or at least 99% pure.

The term “mammal” includes both human and non-human mammals. Similarly, the term “subject” includes both human and veterinary subjects.

A “nucleic acid molecule” is a polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. Such polynucleotides can be synthesized, for example, using an automated DNA synthesizer. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.” With regard to nucleotide molecules, the term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

In addition, a “recombinant nucleic acid” refers to a nucleic acid having nucleotide sequences that are not naturally joined together. This includes nucleic acid vectors comprising an amplified or assembled nucleic acid which can be used to transform a suitable host cell. A host cell that comprises the recombinant nucleic acid is referred to as a “recombinant host cell.” The gene is then expressed in the recombinant host cell to produce, such as a “recombinant polypeptide.” A recombinant nucleic acid may serve a non-coding function (such as a promoter, origin of replication, ribosome-binding site, etc.) as well.

For sequence comparison of nucleic acid sequences, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are used. Methods of alignment of sequences for comparison are well known in the art. Optimal alignment of sequences for comparison can be conducted, for example, by the local homology algorithm of Smith & Waterman, Adv. Appl. Math. 2:482, 1981, by the homology alignment algorithm of Needleman & Wunsch, J. Mol. Biol. 48:443, 1970, by the search for similarity method of Pearson & Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see for example, Current Protocols in Molecular Biology (Ausubel et al., eds 1995 supplement)).

One example of a useful algorithm is PILEUP. PILEUP uses a simplification of the progressive alignment method of Feng & Doolittle, J. Mol. Evol. 35:351-360, 1987. The method used is similar to the method described by Higgins & Sharp, CABIOS 5:151-153, 1989. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, such as version 7.0 (Devereaux et al., Nuc. Acids Res. 12:387-395, 1984.

Another example of algorithms that are suitable for determining percent sequence identity and sequence similarity are the BLAST and the BLAST 2.0 algorithm, which are described in Altschul et al., J. Mol. Biol. 215:403-410, 1990 and Altschul et al., Nucleic Acids Res. 25:3389-3402, 1977. Software for performing BLAST analyses is publicly available through the National Center for Biotechnology Information (http://www.ncbi.nlm.nih.gov/). The BLASTN program (for nucleotide sequences) uses as defaults a word length (W) of 11, alignments (B) of 50, expectation (E) of 10, M=5, N=−4, and a comparison of both strands. The BLASTP program (for amino acid sequences) uses as defaults a word length (W) of 3, and expectation (E) of 10, and the BLOSUM62 scoring matrix (see Henikoff & Henikoff, Proc. Natl. Acad. Sci. USA 89:10915, 1989).

A first nucleic acid sequence is “operably linked” with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

A “pharmaceutically acceptable carrier” of use includes conventional excipients. Remington's Pharmaceutical Sciences, by E. W. Martin, Mack Publishing Co., Easton, Pa., 15th Edition (1975), describes compositions and formulations suitable for pharmaceutical delivery of the fusion proteins herein disclosed. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

A “polypeptide” or “protein” is a polymer in which the monomers are amino acid residues that are joined together through amide bonds. When the amino acids are alpha-amino acids, either the L-optical isomer or the D-optical isomer can be used, the L-isomers being preferred. The terms “polypeptide” or “protein” as used herein is intended to encompass any amino acid sequence and include modified sequences such as glycoproteins. The term “polypeptide” is specifically intended to cover naturally occurring proteins, as well as those that are recombinantly or synthetically produced. A “therapeutic protein” is a protein that, when expressed in a subject, results in an improvement of a sign or a symptom of a particular disorder in that subject. In some embodiments, administration of a “therapeutic protein” results in an improvement of a sign or a symptom of a pancreatic disorder in a subject, such as, but not limited to, diabetes type 1, diabetes type 2, insulinoma, pancreatitis, or pancreatic cancer.

The term “polypeptide fragment” refers to a portion of a polypeptide which exhibits at least one useful epitope. The term “functional fragments of a polypeptide” refers to all fragments of a polypeptide that retain an activity of the polypeptide. Biologically functional fragments, for example, can vary in size from a polypeptide fragment as small as an epitope capable of binding an antibody molecule to a large polypeptide capable of participating in the characteristic induction or programming of phenotypic changes within a cell. An “epitope” is a region of a polypeptide capable of binding an immunoglobulin generated in response to contact with an antigen. Thus, smaller peptides containing the biological activity of insulin, or conservative variants of the insulin, are thus included as being of use.

The term “substantially purified polypeptide” as used herein refers to a polypeptide which is substantially free of other proteins, lipids, carbohydrates or other materials with which it is naturally associated. In one embodiment, the polypeptide is at least 50%, for example at least 80% free of other proteins, lipids, carbohydrates or other materials with which it is naturally associated. In another embodiment, the polypeptide is at least 90% free of other proteins, lipids, carbohydrates or other materials with which it is naturally associated. In yet another embodiment, the polypeptide is at least 95% free of other proteins, lipids, carbohydrates or other materials with which it is naturally associated.

“Preventing” a disease (such as diabetes or a tumor) refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

A “promoter” refers to an array of nucleic acid control sequences which direct transcription of a nucleic acid. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et al., Methods in Enzymology 153:516-544, 1987).

A “recombinant nucleic acid molecule” refers to one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, such as by genetic engineering techniques. Similarly, a recombinant protein is one encoded for by a recombinant nucleic acid molecule. In addition, a recombinant virus is a virus comprising sequence (such as genomic sequence) that is non-naturally occurring or made by artificial combination of at least two sequences of different origin. The term “recombinant” also includes nucleic acids, proteins and viruses that have been altered solely by addition, substitution, or deletion of a portion of a natural nucleic acid molecule, protein or virus. As used herein, “recombinant AAV” refers to an AAV particle in which a recombinant nucleic acid molecule (such as a recombinant nucleic acid molecule encoding a therapeutic protein) has been package

A “subject” refers to any mammal, such as humans, non-human primates, pigs, sheep, cows, rodents and the like which is to be the recipient of the particular treatment. In two non-limiting examples, a subject is a human subject or a non-human primate subject.

A “therapeutically effective amount” refers to a quantity of a specified pharmaceutical or therapeutic agent (e.g. a recombinant AAV) sufficient to achieve a desired effect in a subject, or in a cell, being treated with the agent, such as increasing insulin production or another desired effect in the pancreas. The effective amount of the agent will be dependent on several factors, including, but not limited to the subject or cells being treated, and the manner of administration of the therapeutic composition.

A virus or vector “transduces” a cell when it transfers nucleic acid into the cell. A cell is “transformed” or “transfected” by a nucleic acid transduced into the cell when the DNA becomes stably replicated by the cell, either by incorporation of the nucleic acid into the cellular genome, or by episomal replication.

Numerous methods of transfection are known to those skilled in the art, such as: chemical methods (e.g., calcium-phosphate transfection), physical methods (e.g., electroporation, microinjection, particle bombardment), fusion (e.g., liposomes), receptor-mediated endocytosis (e.g., DNA-protein complexes, viral envelope/capsid-DNA complexes) and by biological infection by viruses such as recombinant viruses {Wolff, J. A., ed, Gene Therapeutics, Birkhauser, Boston, USA (1994)}. In the case of infection by viruses, the infecting virus particles are absorbed by the target cells, resulting in integration of the viral genome into the cellular DNA. Methods for the introduction of genes into the pancreatic endocrine cells are known (e.g. see U.S. Pat. No. 6,110,743, herein incorporated by reference).

Genetic modification of the target cell is an indicium of successful transfection. “Genetically modified cells” refers to cells whose genotypes have been altered as a result of cellular uptakes of exogenous nucleotide sequence by transfection. A reference to a transfected cell or a genetically modified cell includes both the particular cell into which a vector or polynucleotide is introduced and progeny of that cell.

A “transgene” refers to an exogenous gene supplied by a vector or a virus, such as an AAV.

The current disclosure also includes variants of the polypeptides disclosed herein. Variant sequences include those sequences wherein one or more peptides or nucleotides of the sequence have been substituted, deleted, and/or inserted.

Additionally, the AAVs disclosed herein may be derived from various serotypes, including combinations of serotypes (e.g., “pseudotyped” AAV) or from various genomes (e.g., single-stranded or self-complementary). In particular embodiments, the AAV vectors disclosed herein may comprise desired proteins or protein variants. A “variant” as used herein, refers to an amino acid sequence that is altered by one or more amino acids. The variant may have “conservative” changes, wherein a substituted amino acid has similar structural or chemical properties, e.g., replacement of leucine with isoleucine. More rarely, a variant may have “nonconservative” changes, e.g., replacement of a glycine with a tryptophan. Analogous minor variations may also include amino acid deletions or insertions, or both.

Adenoviral vectors and/or adeno-associated viral vectors can be used in the methods disclosed herein. AAV belongs to the family Parvoviridae and the genus Dependovirus. AAV is a small, non-enveloped virus that packages a linear, single-stranded DNA genome. Both sense and antisense strands of AAV DNA are packaged into AAV capsids with equal frequency. In some embodiments the AAV DNA includes a nucleic acid encoding a therapeutic protein.

The AAV genome is characterized by two inverted terminal repeats (ITRs) that flank two open reading frames (ORFs). In the AAV2 genome, for example, the first 125 nucleotides of the ITR are a palindrome, which folds upon itself to maximize base pairing and forms a T-shaped hairpin structure. The other 20 bases of the ITR, called the D sequence, remain unpaired. The ITRs are cis-acting sequences important for AAV DNA replication; the ITR is the origin of replication and serves as a primer for second-strand synthesis by DNA polymerase. The double-stranded DNA formed during this synthesis, which is called replicating-form monomer, is used for a second round of self-priming replication and forms a replicating-form dimer. These double-stranded intermediates are processed via a strand displacement mechanism, resulting in single-stranded DNA used for packaging and double-stranded DNA used for transcription. Located within the ITR are the Rep binding elements and a terminal resolution site (TRS). These features are used by the viral regulatory protein Rep during AAV replication to process the double-stranded intermediates. In addition to their role in AAV replication, the ITR is also essential for AAV genome packaging, transcription, negative regulation under non-permissive conditions, and site-specific integration (Daya and Berns, Clin Microbiol Rev 21(4):583-593, 2008). In some embodiments, these elements are included in the AAV vector.

The left ORF of AAV contains the Rep gene, which encodes four proteins—Rep78, Rep 68, Rep52 and Rep40. The right ORF contains the Cap gene, which produces three viral capsid proteins (VP1, VP2 and VP3). The AAV capsid contains 60 viral capsid proteins arranged into an icosahedral symmetry. VP1, VP2 and VP3 are present in a 1:1:10 molar ratio (Daya and Berns, Clin Microbiol Rev 21(4):583-593, 2008). In some embodiments, these elements are included in the AAV vector. The AAV vector is packaged in the capsid proteins, which can target specific cell types, such as the duct cells, exocrine cells, and endocrine cells.

In some embodiments, a recombinant adeno-associated virus (rAAV) is generated having a capsid of interest. In AAV, the capsid includes VP1, VP2, and VP3. In some non-limiting examples, to produce a vector, a host cell which can be cultured that contains a nucleic acid sequence encoding an adeno-associated virus (AAV) capsid protein of interest, or fragment thereof, as disclosed herein; a functional rep gene; a minigene composed of, at a minimum, AAV inverted terminal repeats (ITRs) and a transgene, such as a transgene encoding a therapeutic protein; and sufficient helper functions to permit packaging in the capsid protein. The components required to be cultured in the host cell to package an AAV minigene in an AAV capsid may be provided to the host cell in trans. Alternatively, any one or more of the required components (e.g., minigene, rep sequences, cap sequences, and/or helper functions) may be provided by a stable host cell which has been engineered to contain one or more of the required components using methods known to those of skill in the art. In some embodiments, a stable host cell will contain the required component(s) under the control of an inducible promoter. However, the required component(s) can be under the control of a constitutive promoter.

In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contains the rep and/or cap proteins under the control of inducible promoters. Still other stable host cells may be generated by one of skill in the art.

The minigene, rep sequences, cap sequences, and helper functions required for producing a rAAV can be delivered to the packaging host cell in the form of any genetic element which transfer the sequences carried thereon. The selected genetic element may be delivered by any suitable method, including those described herein. The methods used to construct vectors are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present invention. See, e.g., K. Fisher et al, J. Virol., 70:520-532 (1993) and U.S. Pat. No. 5,478,745.

The AAV vectors disclosed herein can include a promoter operably linked to a therapeutic protein of interest. In some embodiments, the promoter is an insulin promoter, such as a human, rat or a mouse insulin promoter. The promoter can also be a glucagon promoter or an amylase promoter.

In some embodiments, the AAV genome is modified to include a gene encoding a selectable marker, which includes, but are not limited to, a protein whose expression can be readily detected such as a fluorescent or luminescent protein or an enzyme that acts on a substrate to produce a colored, fluorescent, or luminescent substance (“detectable markers”). There are other genes of use, such as genes that encode drug resistance or provide a function that can be used to purify cells. Selectable markers include neomycin resistance gene (neo), puromycin resistance gene (puro), guanine phosphoribosyl transferase (gpt), dihydrofolate reductase (DHFR), adenosine deaminase (ada), puromycin-N-acetyltransferase (PAC), hygromycin resistance gene (hyg), multidrug resistance gene (mdr), thymidine kinase (TK), hypoxanthine-guanine phosphoribosyltransferase (HPRT), and hisD gene. Detectable markers include green fluorescent protein (GFP) blue, sapphire, yellow, red, orange, and cyan fluorescent proteins and variants of any of these. Luminescent proteins such as luciferase (e.g., firefly or Renilla luciferase) are also selectable makers.

Polypeptide sequences of the current disclosure can also be defined in terms of particular identity and/or similarity with certain polypeptides described herein. The sequence identity will typically be greater than 60%, preferably greater than 75%, more preferably greater than 80%, even more preferably greater than 90%, and can be greater than 95%. The identity and/or similarity of a sequence can be 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% as compared to a sequence disclosed herein. The sequence identify can be about 95%, 96%, 97% 98%, or 99%. Unless otherwise specified, as used herein percent sequence identity and/or similarity of two sequences can be determined using the algorithm of Karlin and Altschul (1990), modified as in Karlin and Altschul (1993). Such an algorithm is incorporated into the NBLAST and XBLAST programs of Altschul et al. (1990). BLAST searches can be performed with the NBLAST program, score=100, wordlength=12, to obtain sequences with the desired percent sequence identity. To obtain gapped alignments for comparison purposes, Gapped BLAST can be used as described in Altschul et al. (1997). When utilizing BLAST and Gapped BLAST programs, the default parameters of the respective programs (NBLAST and XBLAST) can be used.

The vectors disclosed herein can contain nucleic acid sequences encoding an intact AAV capsid which may be from a single AAV serotype.

In an embodiment, the methods may comprise an adeno-associated virus (AAV) vector as the gene therapy agent. The AAV vector can transduce one or more pancreatic cell types, such as islet cells, duct cells, and exocrine (acinar) cells. In the case the AAV vector transduces islet cells or endocrine cells, the AAV vector comprises a capsid protein comprising a capsid sequence selected from a group of AAVDJ (SEQ ID NO: 21), AAVKP1 (SEQ ID NO: 39), AAV2.7m8 (SEQ ID NO: 34), AAV10 (SEQ ID NO: 10), AAVsh10 (SEQ ID NO: 35), AAV1 (SEQ ID NO: 1), AAV7 (SEQ ID NO: 7), AAV2i8 (SEQ ID NO: 22), AAVPHPeB (SEQ ID NO: 28), AAVhu37 (SEQ ID NO: 16), AAVAnc80 (SEQ ID NO: 33), AAVrh8 (SEQ ID NO: 12), AAVLK03 (SEQ ID NO: 20), AAVKP3 (SEQ ID NO: 41), AAV2G9 (SEQ ID NO: 24), and AAVPHPB (SEQ ID NO: 27), or a variant thereof exhibiting at least 70% identity thereto. The sequence identify can be about 95%, 96%, 97% 98%, or 99%. In the case the AAV vector transduces duct cells, the AAV vector comprises a capsid protein comprising a capsid sequence selected from a group of AAV2G9 (SEQ ID NO: 24), AAVKP1 (SEQ ID NO: 39), AAV7 (SEQ ID NO: 7), AAVsh10 (SEQ ID NO: 35), AAV1 (SEQ ID NO: 1), AAVrh20 (SEQ ID NO: 17), AAVKP3 (SEQ ID NO: 41), AAVrh10 (SEQ ID NO: 13), AAVLK03 (SEQ ID NO: 20), AAVDJ (SEQ ID NO: 21), AAV2 (SEQ ID NO: 2), AAVhu37 (SEQ ID NO: 16), AAVNP40 (SEQ ID NO: 25), AAVrh8 (SEQ ID NO: 12), AAV10 (SEQ ID NO: 10), AAV8 (SEQ ID NO: 8), AAVhu13 (SEQ ID NO: 15), AAVAnc80 (SEQ ID NO: 33), and AAVPHPeB (SEQ ID NO: 28), or a variant thereof exhibiting at least 70% identity thereto. The sequence identify can be about 95%, 96%, 97% 98%, or 99%. In the case the AAV vector transduces exocrine (acinar) cells or exocrine cells, the AAV vector comprises a capsid protein comprising a capsid sequence selected from a group of AAVDJ (SEQ ID NO: 21), AAVhu37 (SEQ ID NO: 16), AAVrh8 (SEQ ID NO: 12), AAVNP40 (SEQ ID NO: 25), AAVPHPB (SEQ ID NO: 27), and AAV7 (SEQ ID NO: 7), or a variant thereof exhibiting at least 70% identity thereto. The sequence identify can be about 95%, 96%, 97% 98%, or 99%. These AAV vectors may transduce the one or more pancreatic cell types with an efficiency that is about 5 times to about 200 times greater or about 40 times to about 200 times greater than that of AAV9 delivered at the delivery point. The AAV vector can transduce one or more pancreatic cell types, namely islet cells, duct cells and exocrine (acinar) cells, with an efficiency of about 5, 10, 15, 20, 25, 30, 35, 40, 45, 50, 55, 60, 65, 70, 75, 80, 85, 90, 95, 100, 125, 150, 175, or 200 times greater than that of AAV9 delivered at the delivery point. Furthermore, the AAV vector may detarget at least one pancreatic cell type, resulting in transduction of the at least one pancreatic cell type less than about 0.25 times that of AAV9 delivered at the delivery point. The AAV vector may detarget at least one pancreatic cell type, resulting in transduction of the at least one pancreatic cell type less than about 0.20, 0.15 or 0.10, or 0.05 times that of AAV9 delivered at the delivery point. Yet further, the AAV vector may disseminate to at least one of the liver, heart, and kidney, at a level less than about 0.1 times that of AAV9 delivered at the delivery point. The AAV vector may disseminate to at least one of the liver, heart, and kidney, at a level less than about 0.09, 0.08, 0.07, 0.06, or 0.05 times that of AAV9 delivered at the delivery point.

In some embodiments, the AAV vector can further comprise a polynucleotide the translation product of which is a protein of therapeutic interest. By way of a nonlimiting example, where the method is used to treat type 1 diabetes, the protein of interest can be an insulin-promoting transcription factor. The AAV vector can include a promoter operably linked to a nucleic acid molecule encoding a therapeutic protein. Promoters include, but are not limited to, the glucagon, insulin or amylase promoter. Therapeutic proteins include any protein of use in treating a pancreatic disorder in a subject, including, but not limited to, type 1 diabetes, type 2 diabetes, pancreatic cancer and insulinoma. Therapeutic proteins include endocrine hormones, transcription factors (including insulin promoting transcription factors), and chemotherapeutic proteins.

The disclosed AAV vectors can be used in a method of gene therapy for a disorder of the pancreas in a subject. The AAV vectors comprise a capsid protein sequence selected from SEQ ID NO: 1, SEQ ID NO: 2, SEQ ID NO: 7, SEQ ID NO: 8, SEQ ID NO: 10, SEQ ID NO: 12, SEQ ID NO: 13, SEQ ID NO: 15, SEQ ID NO: 16, SEQ ID NO: 17, SEQ ID NO: 21, SEQ ID NO: 22, SEQ ID NO: 23, SEQ ID NO: 26, SEQ ID NO: 27, SEQ ID NO: 29, SEQ ID NO: 35, SEQ ID NO: 38, SEQ ID NO: 43, and SEQ ID NO: 45, or a variant thereof exhibiting at least 70% identity thereto. The sequence identify can be about 95%, 96%, 97% 98%, or 99%. SEQ ID NOs: 1-45 are the amino acid sequence of VP1 proteins.

The disclosed AAV can be delivered to the pancreas of a subject by any method. One exemplary method for intraductal administration is Endoscopic Retrograde Cholangiopancreatography (ERCP). ERCP is an endoscopic technique that involves the placement of a side-viewing instrument (generally either an endoscope or duodenoscope) within the descending duodenum. The procedure eliminates the need for invasive surgical procedures for administration to the pancreatic duct.

In an ERCP procedure, the patient will generally lie on their side on an examining table. The patient will then be given medication to help numb the back of the patient's throat, and a sedative to help the patient relax during the examination. The patient then swallows the endoscope. The thin, flexible endoscope is passed carefully through the alimentary canal of the patient. The physician guides the endoscope through the patient's esophagus, stomach, and the first part of the small intestine known as the duodenum. Because of the endoscope's relatively small diameter, most patients can tolerate the unusualness of having the endoscope advanced through the opening of their mouth.

The physician stops the advancement of the endoscope when the endoscope reaches the junction where the ducts of the biliary tree and pancreas open into the duodenum. This location is called the papilla of Vater, or also commonly referred to as the ampulla of Vater. The papilla of Vater is a small mound of tissue looking and acting similarly to a nipple. The papilla of Vater emits a substance known as bile into the small intestine, as well as pancreatic secretions that contain digestive enzymes. Bile is a combination of chemicals made in the liver and is necessary in the act of digestion. Bile is stored and concentrated in the gallbladder between meals. When digestive indicators stimulate the gallbladder, however, the gallbladder squeezes the bile through the common bile duct and subsequently through the papilla of Vater. The pancreas secretes enzymes in response to a meal, and the enzymes help digest carbohydrates, fats, and proteins.

The patient is instructed (or manually maneuvered) to lie flat on their stomach once the endoscope reaches the papilla of Vater. For visualization or treatment within the biliary tree, the distal end of the endoscope can be positioned proximate the papilla of Vater. A catheter is then advanced through the endoscope until the distal tip of the catheter emerges from the opening at the endoscope's distal end. The distal end of the catheter is guided through the endoscope's orifice to the papilla of Vater (located between the sphincter of Oddi) leading to the common bile duct and the pancreatic duct. In the case of pancreas-specific delivery of reagents, the pancreatic duct proper can be entered.

ERCP catheters can be constructed from Teflon, polyurethane and polyaminde. ERCP catheters also can also be constructed from resin comprised of nylon and PEBA (see U.S. Pat. No. 5,843,028), and can be construed for use by a single operator (see U.S. Pat. No. 7,179,252). At times, a spring wire guide may be placed in the lumen of the catheter to assist in cannulation of the ducts. A stylet, used to stiffen the catheter, must first be removed prior to spring wire guide insertion.

A dual or multi-lumen ERCP catheter in which one lumen could be utilized to accommodate the spring wire guide or diagnostic or therapeutic device, and in which a second lumen could be utilized for contrast media and/or dye infusion and or for administration of a pharmaceutical composition including a viral vector, such an adenoviral vector, encoding a therapeutic protein. In some embodiments, a contrast dye is administered in addition to the pharmaceutical composition including a disclosed AAV vector. The contrast dye can be a low-osmolar low-viscosity non-ionic dye, a low-viscosity high-osmolar dye, or a dissociable high-viscosity dye. In specific non-limiting examples the dye is lopromid, loglicinate, or loxaglinate. Endoscopes have been designed for the delivery of more than one liquid solution, such as a first liquid composition including a viral vector, such an AAV vector, and a second liquid composition including dye, see U.S. Pat. No. 7,597,662, which is incorporated herein by reference. Thus, the pharmaceutical composition including a viral vector, such an AAV vector, and the dye can be delivered in the same or separate liquid compositions. Methods and devices for using biliary catheters for accessing the biliary tree for ERCP procedures are disclosed in U.S. Pat. Nos. 5,843,028, 5,397,302 5,320,602.

In additional examples, the vector is administered using a viral infusion technique into a pancreatic duct.

AAV virions can be provided as lyophilized preparations and diluted in a stabilizing compositions for immediate or future use. Alternatively, the AAV virions can be provided immediately after production and stored for future use.

Disclosed herein are methods of delivering a gene therapy agent to pancreatic cells and AAV vectors for use in a method of gene therapy for a disorder of the pancreas in a subject. In embodiment, methods are disclosed that comprise guiding a distal end of a catheter to a major duodenal papilla in a duodenum of a subject and advancing the distal end in a retrograde direction through a pancreatic duct to a delivery point internal to a pancreas of the subject. The methods further comprise delivering a therapeutically effective amount of a gene therapy agent through the catheter to the delivery point at an infusion pressure and allowing the gene therapy agent to reside at the delivery point for a dwell time. The distal end of the catheter has a diameter selected to prevent backflow of the gene therapy agent.

The delivery point may be located at various parts of the pancreas. In an embodiment, the delivery point is located distal to the head of the pancreas. More specifically, the delivery point may be located in the body of the pancreas or in the tail of the pancreas.

The dwell time to allowing the gene therapy agent to reside at the delivery point can vary. In an embodiment, the dwell time is from about 5 minutes to about 30 minutes. More specifically, the dwell time can be from about 10 minutes to about 20 minutes.

The step of guiding the distal end of a catheter to the major duodenal papilla can further comprise accessing the duodenum via endoscopy or via surgery. The surgery may be duodenotomy.

In additional embodiments, the method further comprises controlling a fluid pressure within the pancreatic duct to reduce incidence of pancreatitis. In some embodiments, this can comprise aspirating an amount of pancreatic fluid from the pancreatic duct before delivering the gene therapy agent.

The method of gene therapy comprises guiding a distal end of a catheter to a major duodenal papilla in a duodenum of the subject and advancing the distal end in a retrograde direction through the pancreatic duct to a delivery point internal to the pancreas of the subject. The method further comprises delivering a therapeutically effective amount of a gene therapy agent comprising the AAV vector through the catheter to the delivery point at an infusion pressure and allowing the gene therapy agent to reside at the delivery point for a dwell time. The distal end has a diameter selected to prevent backflow of the gene therapy agent, for example by wedging the catheter within the pancreatic duct.

In embodiments, the AAV vector transduces one or more of islet cells, duct cells, and exocrine (acinar) cells with an efficiency that is about 5 times to about 200 times greater than that of AAV9 delivered at the delivery point. More specifically, the efficiency may be about 40 times to about 200 times greater than that of AAV9 delivered at the delivery point. In the case the AAV vector transduces islet cells or endocrine cells, the AAV vector comprises a capsid protein comprising a sequence selected from a group of AAVDJ (SEQ ID NO:21), AAVKP1 (SEQ ID NO: 39), AAV2.7m8 (SEQ ID NO: 34), AAV10 (SEQ ID NO: 10), AAVsh10 (SEQ ID NO: 35), AAV1 (SEQ ID NO: 1), AAV7 (SEQ ID NO: 7), AAV2i8 (SEQ ID NO: 22), AAVPHPeB (SEQ ID NO: 28), AAVhu37 (SEQ ID NO: 16), AAVAnc80 (SEQ ID NO: 33), AAVrh8 (SEQ ID NO: 12), AAVLK03 (SEQ ID NO: 20), AAVKP3 (SEQ ID NO: 41), AAV2G9 (SEQ ID NO: 24), and AAVPHPB (SEQ ID NO: 27), or a variant thereof exhibiting at least 70% identity thereto. The sequence identify can be about 95%, 96%, 97% 98%, or 99%. In the case the AAV vector transduces duct cells, the AAV vector comprises a capsid protein sequence selected from a group of AAV2G9 (SEQ ID NO: 24), AAVKP1 (SEQ ID NO: 39), AAV7 (SEQ ID NO: 7), AAVsh10 (SEQ ID NO: 35), AAV1 (SEQ ID NO: 1), AAVrh20 (SEQ ID NO: 17), AAVKP3 (SEQ ID NO: 41), AAVrh10 (SEQ ID NO: 13), AAVLK03 (SEQ ID NO: 20), AAVDJ (SEQ ID NO: 21), AAV2 (SEQ ID NO: 2), AAVhu37 (SEQ ID NO: 16), AAVNP40 (SEQ ID NO: 25), AAVrh8 (SEQ ID NO: 12), AAV10 (SEQ ID NO: 10), AAV8 (SEQ ID NO: 8), AAVhu13 (SEQ ID NO: 15), AAVAnc80 (SEQ ID NO: 33), and AAVPHPeB (SEQ ID NO: 28), or a variant thereof exhibiting at least 70% identity thereto. The sequence identify can be about 95%, 96%, 97% 98%, or 99%. In the case the AAV vector transduces exocrine (acinar) cells or exocrine cells, the AAV vector comprises a capsid protein comprising a sequence selected from a group of AAVDJ (SEQ ID NO: 21), AAVhu37 (SEQ ID NO: 16), AAVrh8 (SEQ ID NO: 12), AAVNP40 (SEQ ID NO: 25), AAVPHPB (SEQ ID NO: 27), and AAV7 (SEQ ID NO: 7), or a variant thereof exhibiting at least 70% identity thereto. The sequence identify can be about 95%, 96%, 97% 98%, or 99%. Furthermore, the AAV vector may detarget at least one pancreatic cell type, resulting in transduction of the at least one pancreatic cell type less than about 0.25 times that of AAV9 delivered at the delivery point. Yet further, the AAV vector may disseminate to at least one of the liver, heart, and kidney, at a level less than about 0.1 times that of AAV9 delivered at the delivery point.

In some embodiments, the pancreatic disorder can comprise type 1 diabetes, cystic fibrosis, hypercalcemia, or hypertriglyceridemia, type 2 diabetes, hereditary pancreatitis, or pancreatic cancer.

In more embodiments, the AAV vector can further comprise a polynucleotide the translation product of which is a protein of therapeutic interest with respect to the pancreatic disorder. By way of a nonlimiting example, where the pancreatic disorder is type 1 diabetes, the protein of interest can be an insulin-promoting transcription factor.

In further embodiments, a method of treating a pancreatic disorder can comprise transducing pancreatic cells of the patient with AAV vectors selected from SEQ ID NO: 1, SEQ ID NO: 7, SEQ ID NO: 10, SEQ ID NO: 12, SEQ ID NO: 16, SEQ ID NO: 20, SEQ ID NO:21, SEQ ID NO: 22, SEQ ID NO: 24, SEQ ID NO: 27, SEQ ID NO: 28, SEQ ID NO: 33, SEQ ID NO: 34, SEQ ID NO: 35, SEQ ID NO: 39, and SEQ ID NO: 41, or a variant thereof exhibiting at least 70% identity thereto. The sequence identify can be about 95%, 96%, 97% 98%, or 99%.

In some embodiments, the AAV vector transduces one or more pancreatic cell types selected from a group of islet cells, duct cells, and exocrine (acinar) cells with an efficiency that is about 5 times to about 200 times greater than that of AAV9 delivered at the delivery point. More specifically, the efficiency may be about 40 times to about 200 times greater than that of AAV9 delivered at the delivery point. In the case the AAV vector transduces islet cells or endocrine cells, the AAV vector comprises a capsid protein comprising a sequence selected from a group of AAVDJ (SEQ ID NO: 21), AAVKP1 (SEQ ID NO: 39), AAV2.7m8 (SEQ ID NO: 34), AAV10 (SEQ ID NO: 10), AAVsh10 (SEQ ID NO: 35), AAV1 (SEQ ID NO: 1), AAV7 (SEQ ID NO: 7), AAV2i8 (SEQ ID NO: 22), AAVPHPeB (SEQ ID NO: 28), AAVhu37 (SEQ ID NO: 16), AAVAnc80 (SEQ ID NO: 33), AAVrh8 (SEQ ID NO: 12), AAVLK03 (SEQ ID NO: 20), AAVKP3 (SEQ ID NO: 41), AAV2G9 (SEQ ID NO: 24), and AAVPHPB (SEQ ID NO: 27), or a variant thereof exhibiting at least 70% identity thereto. The sequence identify can be about 95%, 96%, 97% 98%, or 99%. In the case the AAV vector transduces duct cells, the AAV vector comprises a capsid protein sequence selected from a group of AAV2G9 (SEQ ID NO: 24), AAVKP1 (SEQ ID NO: 39), AAV7 (SEQ ID NO: 7), AAVsh10 (SEQ ID NO: 35), AAV1 (SEQ ID NO: 1), AAVrh20 (SEQ ID NO: 17), AAVKP3 (SEQ ID NO: 41), AAVrh10 (SEQ ID NO: 13), AAVLK03 (SEQ ID NO: 20), AAVDJ (SEQ ID NO: 21), AAV2 (SEQ ID NO: 2), AAVhu37 (SEQ ID NO: 16), AAVNP40 (SEQ ID NO: 25), AAVrh8 (SEQ ID NO: 12), AAV10 (SEQ ID NO: 10), AAV8 (SEQ ID NO: 8), AAVhu13 (SEQ ID NO: 15), AAVAnc80 (SEQ ID NO: 33), and AAVPHPeB (SEQ ID NO: 28), or a variant thereof exhibiting at least 70% identity thereto. The sequence identify can be about 95%, 96%, 97% 98%, or 99%. In the case the AAV vector transduces exocrine (acinar) cells or exocrine cells, the AAV vector comprises a capsid protein comprising a sequence selected from a group of AAVDJ (SEQ ID NO: 21), AAVhu37 (SEQ ID NO: 16), AAVrh8 (SEQ ID NO: 12), AAVNP40 (SEQ ID NO: 25), AAVPHPB (SEQ ID NO: 27), and AAV7 (SEQ ID NO: 7), or a variant thereof exhibiting at least 70% identity thereto. The sequence identify can be about 95%, 96%, 97% 98%, or 99%. Furthermore, the AAV vector may detarget at least one pancreatic cell type, resulting in transduction of the at least one pancreatic cell type less than about 0.25 times that of AAV9 delivered at the delivery point. Yet further, the AAV vector may disseminate to at least one of the liver, heart, and kidney, at a level less than about 0.1 times that of AAV9 delivered at the delivery point.

In an embodiment, the pancreatic disorder can comprise type 1 diabetes, type 2 diabetes, hereditary pancreatitis, or pancreatic cancer.

In an embodiment, the AAV vector can further comprise a polynucleotide the translation product of which is a protein of therapeutic interest with respect to the pancreatic disorder. By way of a nonlimiting example, where the pancreatic disorder is type 1 diabetes, the protein of interest can be an insulin-promoting transcription factor.

While AAV9 is presented in various embodiments described above as a benchmark for characteristics or properties that are exceeded by other AAV vectors, it should be understood that AAV9 and other capsids comprising a capsid protein comprising SEQ ID NO: 9 or variants thereof may also be used in the methods described herein.

The pharmaceutical compositions can be produced contain the AAV and a pharmaceutically acceptable excipient. Such excipients include any pharmaceutical agent that does not itself induce the production of antibodies harmful to the individual receiving the composition, and which may be administered without undue toxicity. Pharmaceutically acceptable excipients include, but are not limited to, liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in REMINGTON'S PHARMACEUTICAL SCIENCES (Mack Pub. Co., N.J. 1991).

In some embodiments, the excipients confer a protective effect on the AAV virion such that loss of AAV virions, as well as transduceability resulting from formulation procedures, packaging, storage, transport, and the like, is minimized. These excipient compositions are therefore considered “virion-stabilizing” in the sense that they provide higher AAV virion titers and higher transduceability levels than their non-protected counterparts, as measured using standard assays, see, for example, Published U.S. Application No. 2012/0219528. These compositions therefore demonstrate “enhanced transduceability levels” as compared to compositions lacking the particular excipients described herein, and are therefore more stable than their non-protected counterparts.

Exemplary excipients that can used to protect an AAV virion from activity degradative conditions include, but are not limited to, detergents, proteins, e.g., ovalbumin and bovine serum albumin, amino acids, e.g., glycine, polyhydric and dihydric alcohols, such as but not limited to polyethylene glycols (PEG) of varying molecular weights, such as PEG-200, PEG-400, PEG-600, PEG-1000, PEG-1450, PEG-3350, PEG-6000, PEG-8000 and any molecular weights in between these values, with molecular weights of 1500 to 6000 preferred, propylene glycols (PG), sugar alcohols, such as a carbohydrate, preferably, sorbitol. The detergent, when present, can be an anionic, a cationic, a zwitterionic or a nonionic detergent. An exemplary detergent is a nonionic detergent. One suitable type of nonionic detergent is a sorbitan ester, e.g., polyoxyethylenesorbitan monolaurate (TWEEN®-20) polyoxyethylenesorbitan monopalmitate (TWEEN®-40), polyoxyethylenesorbitan monostearate (TWEEN®-60), polyoxyethylenesorbitan tristearate (TWEEN®-65), polyoxyethylenesorbitan monooleate (TWEEN®-80), polyoxyethylenesorbitan trioleate (TWEEN®-85), such as TWEEN®-20 and/or TWEEN®-80. These excipients are commercially available from a number of vendors, such as Sigma, St. Louis, Mo.

The amount of the various excipients present in any of the disclosed compositions varies and is readily determined by one of skill in the art. For example, a protein excipient, such as BSA, if present, will can be present at a concentration of between 1.0 weight (wt.) % to about 20 wt. %, preferably 10 wt. %. If an amino acid such as glycine is used in the formulations, it can be present at a concentration of about 1 wt. % to about 5 wt. %. A carbohydrate, such as sorbitol, if present, can be present at a concentration of about 0.1 wt % to about 10 wt. %, such as between about 0.5 wt. % to about 15 wt. %, or about 1 wt. % to about 5 wt. %. If polyethylene glycol is present, it can generally be present on the order of about 2 wt. % to about 40 wt. %, such as about 10 wt. % top about 25 wt. %. If propylene glycol is used in the subject formulations, it will typically be present at a concentration of about 2 wt. % to about 60 wt. %, such as about 5 wt. % to about 30 wt. %. If a detergent such as a sorbitan ester (TWEEN®) is present, it can be present at a concentration of about 0.05 wt. % to about 5 wt. %, such as between about 0.1 wt. % and about 1 wt %, see U.S. Published Patent Application No. 2012/0219528, which is incorporated herein by reference. In one example, an aqueous virion-stabilizing formulation comprises a carbohydrate, such as sorbitol, at a concentration of between 0.1 wt. % to about 10 wt. %, such as between about 1 wt. % to about 5 wt. %, and a detergent, such as a sorbitan ester (TWEEN®) at a concentration of between about 0.05 wt. % and about 5 wt. %, such as between about 0.1 wt. % and about 1 wt. %. Virions are generally present in the composition in an amount sufficient to provide a therapeutic effect when given in one or more doses, as defined above.

The pharmaceutical compositions can include a contrast dye is administered in addition to the viral vector, such an adenoviral vector, including an insulin promoter operably linked to a nucleic acid molecule encoding Pdx1 and MafA. The contrast dye can be a low-osmolar low-viscosity non-ionic dye, a low-viscosity high-osmolar dye, or a dissociable high-viscosity dye. In specific non-limiting examples, the dye is lopromid, loglicinate, or loxaglinate.

Appropriate doses depend on the subject being treated (e.g., human or nonhuman primate or other mammal), age and general condition of the subject to be treated, the severity of the condition being treated, the mode of administration of the AAV vector/virion, among other factors. An appropriate effective amount can be readily determined by one of skill in the art. Thus, a “therapeutically effective amount” will fall in a relatively broad range that can be determined through clinical trials. The method can include measuring an outcome, such as insulin production, improvement in a fasting plasma glucose tolerance test, or pancreatic beta cell number. The method can include administering other therapeutic agents, such as insulin. The method can also include having the subject make lifestyle modifications.

For example, for in vivo injection, i.e., injection directly to the subject, a therapeutically effective dose will be on the order of from about 10⁵ to 10¹⁶ of the AAV virions, such as 10⁸ to 10¹⁴ AAV virions. The dose, of course, depends on the efficiency of transduction, promoter strength, the stability of the message and the protein encoded thereby, and clinical factors. Effective dosages can be readily established by one of ordinary skill in the art through routine trials establishing dose response curves.

Dosage treatment may be a single dose schedule or a multiple dose schedule to ultimately deliver the amount specified above. Moreover, the subject may be administered as many doses as appropriate. Thus, the subject may be given, e.g., 10⁵ to 10¹⁶ AAV virions in a single dose, or two, four, five, six or more doses that collectively result in delivery of, e.g., 10⁵ to 10¹⁶ AAV virions. One of skill in the art can readily determine an appropriate number of doses to administer.

In some embodiments, the AAV is administered at a dose of about 1×10¹¹ to about 1×10¹⁴ viral particles (vp)/kg. In some examples, the AAV is administered at a dose of about 1×10¹² to about 8×10¹³ vp/kg. In other examples, the AAV is administered at a dose of about 1×10¹³ to about 6×10¹³ vp/kg. In specific non-limiting examples, the AAV is administered at a dose of at least about 1×10¹¹, at least about 5×10¹¹, at least about 1×10¹², at least about 5×10¹², at least about 1×10¹³, at least about 5×10¹³, or at least about 1×10¹⁴ vp/kg. In other non-limiting examples, the AAV is administered at a dose of no more than about 5×10¹¹, no more than about 1×10¹², no more than about 5×10¹², no more than about 1×10¹³, no more than about 5×10¹³, or no more than about 1×10¹⁴ vp/kg. In one non-limiting example, the AAV is administered at a dose of about 1×1012 vp/kg. The AAV can be administered in a single dose, or in multiple doses (such as 2, 3, 4, 5, 6, 7, 8, 9 or 10 doses) as needed for the desired therapeutic results.

### EXAMPLES

### Example 1—Generating and Preparing AAV vectors

The AAV vectors used in the experiments were: AAV9 vector expressing TdTomato under the control of the CAG promoter (AAV9-CAG-TdTomato), AAVKP1 vector¹ expressing TdTomato under the control of the CAG promoter (AAVKP1-CAG-TdTomato), and DNA/RNA-barcoded AAV capsid library expressing DNA barcodes (BCs) as RNA barcode transcripts under the control of the CAG promoter (AAVx-CAG-BC Library). The AAVx-CAG-BC Library contains 36 AAV capsid strains as summarized in Table 1. The AAVx-CAG-BC Library was used for the AAV Barcode-Seq analysis to determine transduction and pharmacokinetic profiles of AAV capsids contained in the library. The AAV Barcode-Seq technology has been described in detail elsewhere²⁻⁵.

TABLE 1
AAV strains contained in AAVx-CAG-BC Library

| AAV ID | Capsid VP1 SEQ ID No. |
|---|---|
| AAV1 | SEQ ID NO: 1 |
| AAV2 | SEQ ID NO: 2 |
| AAV3 | SEQ ID NO: 3 |
| AAV4 | SEQ ID NO: 4 |
| AAV5 | SEQ ID NO: 5 |
| AAV6 | SEQ ID NO: 6 |
| AAV7 | SEQ ID NO: 7 |
| AAV8 | SEQ ID NO: 8 |
| AAV9 | SEQ ID NO: 9 |
| AAV10 | SEQ ID NO: 10 |
| AAV11 | SEQ ID NO: 11 |
| AAVrh8 | SEQ ID NO: 12 |
| AAVrh10 | SEQ ID NO: 13 |
| AAVhu11 | SEQ ID NO: 14 |
| AAVhu13 | SEQ ID NO: 15 |
| AAVhu37 | SEQ ID NO: 16 |
| AAVrh20 | SEQ ID NO: 17 |
| AAVrh43 | SEQ ID NO: 18 |
| AAVbb2 | SEQ ID NO: 19 |
| AAVLK03 | SEQ ID NO: 20 |
| AAVDJ | SEQ ID NO: 21 |
| AAV2i8 | SEQ ID NO: 22 |
| AAVR585E | SEQ ID NO: 23 |
| AAV2G9 | SEQ ID NO: 24 |
| AAVNP40 | SEQ ID NO: 25 |
| AAVNP59 | SEQ ID NO: 26 |
| AAVPHPB | SEQ ID NO: 27 |
| AAVPHPeB | SEQ ID NO: 28 |
| AAVPHPS | SEQ ID NO: 29 |
| AAVAnc80 | SEQ ID NO: 33 |
| AAV2.7m8 | SEQ ID NO: 34 |
| AAVsh10 | SEQ ID NO: 35 |
| AAV2retro | SEQ ID NO: 38 |
| AAVKP1 | SEQ ID NO: 39 |
| AAVKP2 | SEQ ID NO: 40 |
| AAVKP3 | SEQ ID NO: 41 |

### Example 2—Testing AAV Capsid Transduction Introduced By Intravenous Injection Method

To identify AAV capsids that can transduce pancreatic cells better than AAV9 by intravenous injection in non-human primates, an AAV DNA/RNA Barcode-Seq study using dsAAVx-U6-VBC Library²⁻⁵ was performed. The dsAAVx-U6-BC viral genomes expressed DNA barcodes (BCs) as RNA barcode transcripts under the control of the human U6 small nuclear RNA (U6 snRNA) promoter as described in detail elsewhere²⁻⁵. A dsAAV-U6-BC virus library comprising 31 AAV strains including 24 strains contained in the library was produced and used (Table 1 and Table 2). This virus library was injected by intravenous injection in two rhesus macaques at a dose of 4×10¹² vg/kg (AS332, female, 11.2 years old, 7.1 kg; AS336, female, 3.8 years old, 5.0 kg). Various tissues including the pancreas were harvested 6 weeks post-injection and subjected to the AAV RNA Barcode-Seq analysis. This study revealed that, except for AAV7 capsid in one rhesus macaque (AS336) showing 6× better transduction in the pancreas than that of AAV9, none of the AAV strains tested in this experiment showed better pancreatic transduction than AAV9 (Table 2). Enhanced AAV7 vector transduction was not observed in AS332, the reason for which has yet to be determined. It may be due to individual-to-individual variations in AAV vector transduction in non-human primates, which were often observed in this study, or due to the presence of anti-AAV neutralizing antibodies against AAV7. The absence of anti-AAV neutralizing antibodies were confirmed only for AAV2 and AAV9 before the injection. With these observations, it may not be possible for one to predict or assume that a pancreatic duct (PD) injection would mediate vector transduction in the pancreatic better than AAV9 by more than one order of magnitude.

TABLE 2
Relative transduction efficiency of various AAV capsids in the
pancreas following intravenous injection in rhesus macaques

Animal ID*

| AAV Strain | AS332 | AS336 |
|---|---|---|
| AAV1 | 0.001 | 0.001 |
| AAV2 | 0.001 | 0.000 |
| AAV3 | 0.001 | 0.001 |
| AAV4 | 0.001 | 0.001 |
| AAV5 | 0.004 | 0.001 |
| AAV6 | 0.002 | 0.001 |
| AAV7 | 0.001 | 5.984 |
| AAV8 | 0.166 | 0.054 |
| AAV9 | 1.000 | 1.000 |
| AAV10 | 0.129 | 0.001 |
| AAV11 | 0.152 | 0.002 |
| AAVrh8 | 0.143 | 0.423 |
| AAVrh10 | 0.134 | 0.001 |
| AAVhu11 | 0.019 | 0.030 |
| AAVhu13 | 0.096 | 0.373 |
| AAVhu37 | 0.049 | 0.724 |
| AAVrh20 | 0.445 | 0.305 |
| AAVrh43 | 0.077 | 0.107 |
| AAVbb2 | 0.006 | 0.002 |
| AAVLK03 | 0.001 | 0.001 |
| AAVDJ | 0.001 | 0.001 |
| AAV2i8 | 0.001 | 0.052 |
| AAV2R585E | 0.002 | 0.001 |
| AAV2G9 | 0.006 | 0.003 |

*These two animals (AS332 and AS336) received dsAAVx-U6-BC virus library intravenously and transduction efficiency of each AAV capsid relative to that of AAV9 was determined by AAV RNA Barcode-Seq. Among the 31 AAV strains' data obtained in this experiment, only that data for 24 AAV strains that were also tested in the PD experiment are shown in this table.
Note:
Values are Phenotypic Difference values obtained by AAV RNA Barcode-Seq analysis representing phenotypic differences compared to the phenotypes of AAV9.

### Example 3—Introducing AAV to Pancreas by Catheter Wedging Method

A total of 9 rhesus macaques with AAV vectors via the pancreatic duct (PD) route (Table 3).

TABLE 3
Summary of AAV vector injection into rhesus macaques
via the pancreatic duct

| Animal | | | Body | | | Incubation |
|---|---|---|---|---|---|---|
| ID | Sex | Age | weight | Vector | Dose | period |
| AS485 | M | 1.5 y | 2.3 kg | AAV9-CAG- | 1.4 × | 4 wk |
| | | | | TdTomato | 10¹³ vg | |
| | | | | | (1.0 ml) | |
| AS510 | F | 3.2 y | 3.6 kg | AAV9-CAG- | 1.4 × | 4 wk |
| | | | | TdTomato | 10¹³ vg | |
| | | | | | (1.0 ml) | |
| AS516 | M | 1.8 y | 3.5 kg | AAVx-CAG- | 8.5 × | 6 wk |
| | | | | BC Library | 10¹² vg | |
| | | | | | (1.0 ml) | |
| AS532 | M | 5.5 y | 9.8 kg | AAVx-CAG- | 8.5 × | 6 wk |
| | | | | BC Library | 10¹² vg | |
| | | | | | (1.0 ml) | |
| AS567 | M | 3.3 y | 5.3 kg | AAVKP1-CAG- | 8.4 × | 4 wk |
| | | | | TdTomato | 10¹² vg | |
| | | | | | (1.2 ml) | |
| AS606 | F | 2.8 y | 3.7 kg | AAVKP1-CAG- | 8.4 × | 4 wk |
| | | | | TdTomato | 10¹² vg | |
| | | | | | (1.2 ml) | |
| AS607 | F | 1.1 y | 2.2 kg | AAVKP1-CAG- | 8.4 × | 4 wk |
| | | | | TdTomato | 10¹² vg | |
| | | | | | (1.2 ml) | |
| AS613 | M | 2.1 y | 3.7 kg | AAVx-CAG-BC | 9.8 × | 6 wk |
| | | | | Library | 10¹² vg | |
| | | | | | (1.5 ml) | |
| AS620 | F | 2.5 y | 3.7 kg | AAVKP1-CAG- | 1.4 × | 4 wk |
| | | | | TdTomato | 10¹² vg | |
| | | | | | (2.0 ml) | |

Injection of AAV vector into the PD was performed under the real-time image guidance in the angiography suite in the Primate Multimodality Imaging Center (PMIC) at the Oregon National Primate Research Center (ONPRC) except for animal AS485. The angiography suite is equipped with a biplane imaging system (Innova 2,000 GE Healthcare). AAV injection for AS485 was done in a standard operating room using a portable x-ray system.

The pancreatic duct (PD) injections for AS510, AS516, AS532, AS606, AS607, AS613 and AS620 were performed in the following manner.

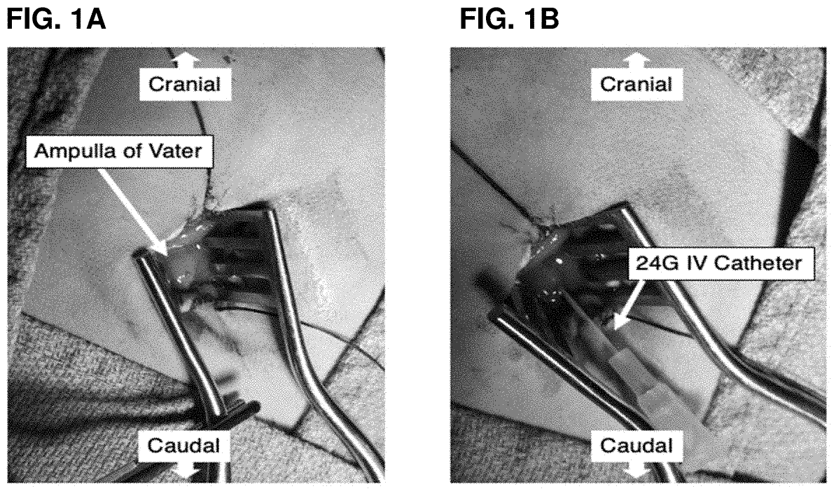

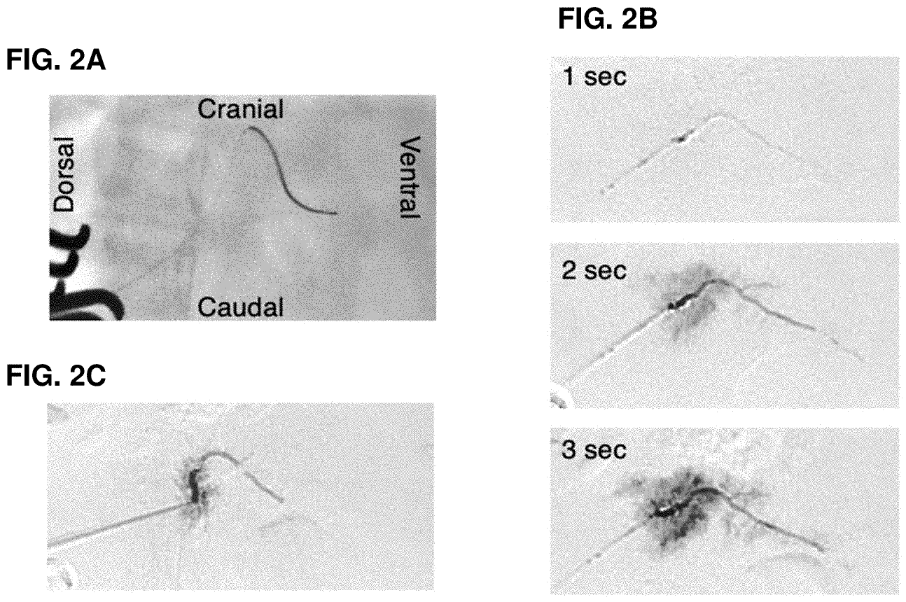

The animals were prescreened for the absence of anti-AAV binding and neutralizing antibodies by ELISA and a cell-based functional assays, respectively, using the methods as previously described⁶. For the PD injection of AAV9-CAG-TdTomato and AAVx-CAG-BC Library, screening was conducted for the absence of anti-AAV2 and anti-AAV9 antibodies. For the injection of AAVKP1-CAG-TdTomato, screening was conducted for the absence of at least anti-AAVKP1 antibodies. The pre-screened animals were intubated and maintained on general anesthesia during the procedure for the PD injection. The animals were positioned in left lateral recumbency followed by sterile preparation and draping of the right caudal thorax and cranial abdomen. A skin incision was made parallel to, and just caudal to, the last rib. Abdominal musculature was then dissected parallel to the muscle fibers in each layer. The abdominal cavity was entered and a Weitlander retractor was placed in the incision. The pylorus of the stomach was visualized and the duodenum was isolated with laparotomy sponges and gauze. Atraumatic forceps or stay sutures were applied to stabilize the duodenum and an approximately 2 cm-long longitudinal antimesenteric duodenotomy incision was made, the ingesta was removed using gauze, and the ampulla of Vater was identified (FIG. 1 panel A). The orifice of the ampulla can be identified as a small wet dot in the center of the ampulla. A 24-gauge (G) angiocatheter (Surflo) was inserted into the cholangio-pancreatic duct system through the orifice of the ampulla of Vater (FIG. 1 panel B), and a small volume of a contrast agent (Iohexol, Omnipaque 300) was injected into the duct system through the angiocatheter to confirm the appropriate anatomy of the pancreatobiliary system. A guidewire is subsequently inserted into the PD through the angiocatheter (not shown). A 0.014-inch guidewire was then navigated to the pancreatic tail side of the main pancreatic duct (FIG. 2 panel A). A 4 or 5-French micropuncture catheter was then advanced into pancreatic head-body junction of the main pancreatic duct over the guidewire. Once the micropuncture catheter engagement was completed, a guidewire was removed and a small amount of the contrast agent was injected into the PD through the micropuncture catheter to confirm the location of the tip of the catheter as well as the wedged condition showing no evidence of leakage of the contrast agent into the proximal portion of the pancreas or the duodenum (FIG. 2 panel B). The time indicated on each image indicates the time after the initiation of injection of the contrast agent. In this procedure, an acinar shadow can be observed. With the conformation of the appropriate positioning and wedged condition of the catheter, 0.5 ml of PBS/5% sorbitol was injected into the PD followed by a 10-min dwelling to allow the infusate to be absorbed into the tissue and the bloodstream and release the pressure. An AAV vector agent in 1.0 to 2.0 ml of PBS/5% sorbitol was slowly injected into the PD via the catheter over 2 minutes immediately followed by injection of 0.15 ml of PBS/5% sorbitol (i.e., 0.1 ml dead space attributed to the micropuncture catheter plus 0.05 ml) to flush the agent out of the catheter. The agent was then dwelled for 20 minutes. Immediately after the dwelling time period is over, a small amount of the contrast agent was injected into the PD to confirm the maintained position (FIG. 2 panel C) and wedged condition of the catheter, and the catheter was removed out of the PD. Upon completion of the injection, the duodenotomy was closed with 4-0 monocryl, followed by the surgical procedure for the standard laparotomy closure. The animals were put under observation on the operation table until extubation. Subsequently, any adverse events including post-procedure pancreatitis were carefully monitored through clinical symptoms and blood tests including serum levels of amylase and lipase.

For the animal AS567, a slightly modified method was used. After successful wedging of a 5-French micropuncture catheter at the head-body junction in the main pancreatic duct, a long 1.7 to 2.4-French microcatheter was inserted and advanced to the pancreatic tail using a guidewire and aspirate pancreatic juice as much as possible to minimize the intrapancreatic duct pressure. In this procedure, an acinar shadow following injection of a small volume of a contrast agent was not observed. Five minutes before AAV vector injection, Octreotide was infused intravenously to suppress pancreatic exocrine secretion. Then the AAV vector agent was slowly injected through the microcatheter over a 2-min period from the upstream side (from the microcatheter tip placed in the tail) toward the downstream, immediately followed by injection of 0.15 ml of PBS/5% sorbitol to flush the agent out of the microcatheter. The agent dwelling time was reduced to 10 min. Blood samples were collected for the complete blood cell count (CBC), chemistry panel including amylase and lipase, and AAV vector genome quantification following injection.

In another experiment on two rhesus macaques negative for anti-AAV9 neutralizing antibodies, a laparotomy was performed, a small incision of the duodenum wall made, the ampulla of Vater directly visualized, and a catheter inserted under real-time fluoroscopy into PD with the catheter tip wedged at the proximal portion of the pancreatic body. Backflow of PD-injected agents from PD to the duodenum can be prevented by selecting an appropriate size of the catheter. Then 1.4×10¹³ vg of AAV9-CAG-tdTomato was injected over a period of 2 min following a 20 min dwelling time period. Blood samples were collected multiple times pre- and post-injection to assess safety and determine pharmacokinetic profiles of PD-injected AAV9 vector. The animals were euthanized 4 weeks post-injection and multiple tissues including the pancreas and the liver were harvested for downstream molecular and histological analyses.

In another experiment on four rhesus macaques negative for anti-AAVKP1 neutralizing antibodies, 8.4×10¹² to 1.4×10¹³ vg of AAVKP1-CAG-tdTomato was injected in the same manner as described above. Blood samples were collected multiple times pre- and post-injection to assess safety and determine pharmacokinetic profiles of PD-injected AAVKP1 vector. The animals were euthanized 4 weeks post-injection and multiple tissues including the pancreas and the liver were harvested for downstream molecular and histological analyses.

### Example 4—Assessing Adverse Effects Due to Contrast Agents and Treatment with AAV

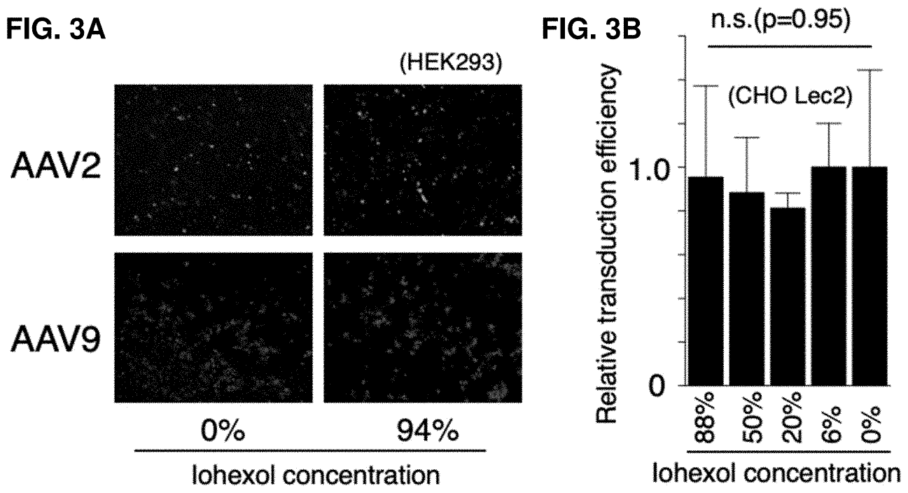

Use of a contrast agent can be useful for successful PD injection of agents. Even though the PD injection procedure described herein has a 10-min interval between injection of a small amount of a contrast agent and injection of an AAV vector, some contrast agent may still remain in the pancreatic tissue, which eventually contact AAV vector particles at PD injection. Therefore, whether the presence of the contrast agents inhibits AAV vector transduction was further investigated. To this end, AAV2 vector or AAV9 vector expressing a fluorescence marker (AAV2-CMV-GFP or AAV9-CAG-TdTomato) were mixed with varying amounts of lohexol (Omnipaque 300). HEK293 cell seeded on a 96-well plate (2×10⁴ cells per well) were exposed to a 25 μL of solution containing 2×10⁹ vg of AAV2-CMV-GFP vector (FIG. 3 panel A upper row) or AAV9-CAG-TdTomato (FIG. 3 panel A bottom row). To make 0% lohexol-containing agents, AAV vectors were diluted with DMEM. To make 94% lohexol-containing agents, AAV vectors were diluted with lohexol (Omnipaque 300). HEK293 cells pre-treated with wild-type adenovirus type 5 at an MOI of 100 were exposed to these AAV vector agents for 30 min, washed with DMEM, and cultured with a complete DMEM for 2 days, and AAV vector transduction was assessed by fluorescence microscopy. It was found that even though HEK293 cells were directly exposed for 30 min to an AAV vector-containing solution made by mixing a 0.94 part of a contrast agent and a 0.06 part of an AAV vector agent (the highest concentration of lohexol in our experimental setting), HEK293 cell transduction efficiency was not affected, demonstrating that high concentrations of lohexol have no effects on AAV transduction (FIG. 3 panel A). This was also confirmed in a separate study in which AAV9-CMV-luciferase vector, lohexol, and CHO Lec2 cells were used (FIG. 3 panel B). CHO Lec2 cell seeded on a 96-well plate (2×10⁴ cells per well) were exposed to a 25 μL of solution containing 2×10⁹ vg of AAV9-CMV-lucifease vector. lohexol-containing agents were prepared in the same way described above; however, the maximum lohexol concentration that was attained was 88% due to the lower titer of the AAV9-CMV-lucifease vector stock. A luciferase assay was performed 2 days post-infection. Y-axis shows relative luciferase activities compared to those obtained by the 0% condition. One-way ANOVA shows no statistically significant difference between the groups (n.s., not significant). Although only two AAV capsids and two cell types were tested, it may reasonably be inferred that the co-presence of the lohexol contrast agent in the pancreatic duct and pancreatic tissue does not have any negative impact on AAV vector transduction.

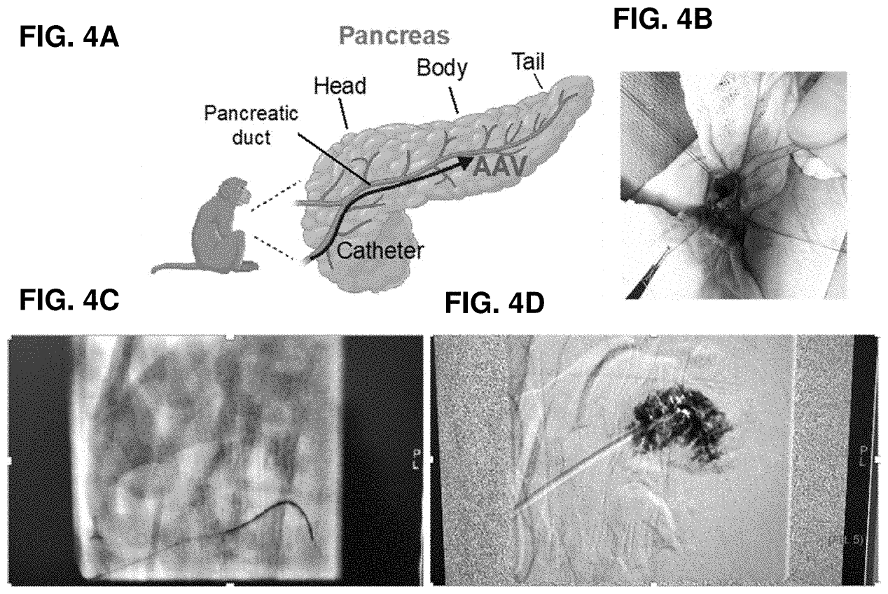

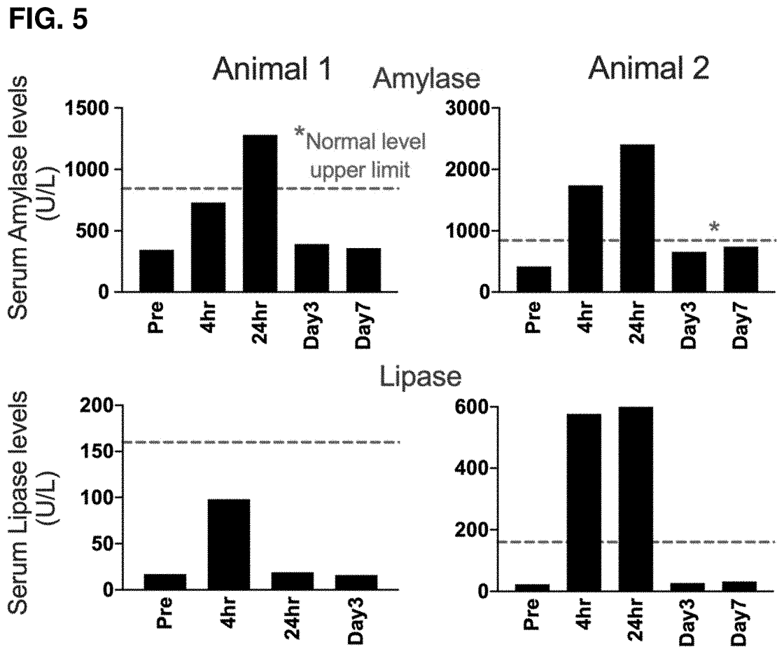

Pancreatitis caused by PD injection procedures were also assessed. A catheter was inserted into pancreatic duct from the ampulla of Vater, and AAV9-CAG-tdTomato vector was infused in body region (FIG. 4). FIG. 4 panel B shows laparotomy where the duodenum wall was opened to directly visualize the ampulla of Vater. A guidewire was inserted into the pancreatic duct under a live-imaging fluoroscopy (FIG. 4 panel C) and an infusion of a contrast agent after AAV infusion was performed for the confirmation of catheter position in body region (FIG. 4 panel d). The animals were evaluated and their tissues were collected after euthanization at 4 weeks post-injection. Amylase and lipase levels in serum were measured at the indicated time points after AAV9 vector injection (FIG. 5). The dotted line in red represents the upper limit of normal amylase and lipase levels. *indicates references for amylase level that are in ONPRC colony.

Although serum amylase and lipase levels were elevated following the injection in all of the nine animals injected with AAV vector, the levels went down to the normal range within 3 days post-injection and no clinical signs suggesting post-procedure pancreatitis were observed. The AAV9 vector leaked into the systemic circulation with the vector concentrations in blood reaching a peak at 8 h post-injection, resulting in vector genome dissemination to non-pancreatic organs to some degree. The AAVKP1 vector also leaked into the systemic circulation but to a much lesser degree compared to AAV9 with the vector concentrations in blood reaching a peak at earlier time points following injection. This results in vector genome dissemination to non-pancreatic organs to a much lesser degree compared to AAV9. In the pancreas, the AAV9 vector transduced many acinar cells and some islets with a few showing good endocrine cell transduction while AAVKP1 showed enhanced islet transduction compared to AAV9. In summary, this study demonstrates that the PD injection is safe and transduces both acinar and islet cells with AAV9 and AAVKP1 vectors. This local approach with AAV9 still allows vector spillover in other non-target organs; however, AAVKP1 can minimize such vector spillover.

### Example 5—Assessing AAV9 and AAVKP1 for Transduction of Pancreatic Islet, Duct Cells and Acinar Cells

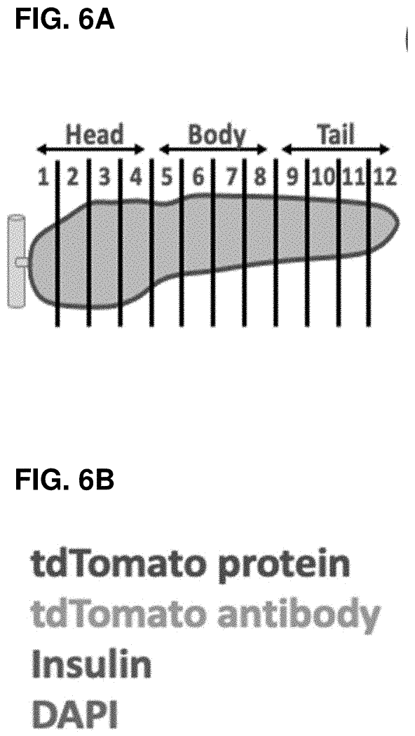

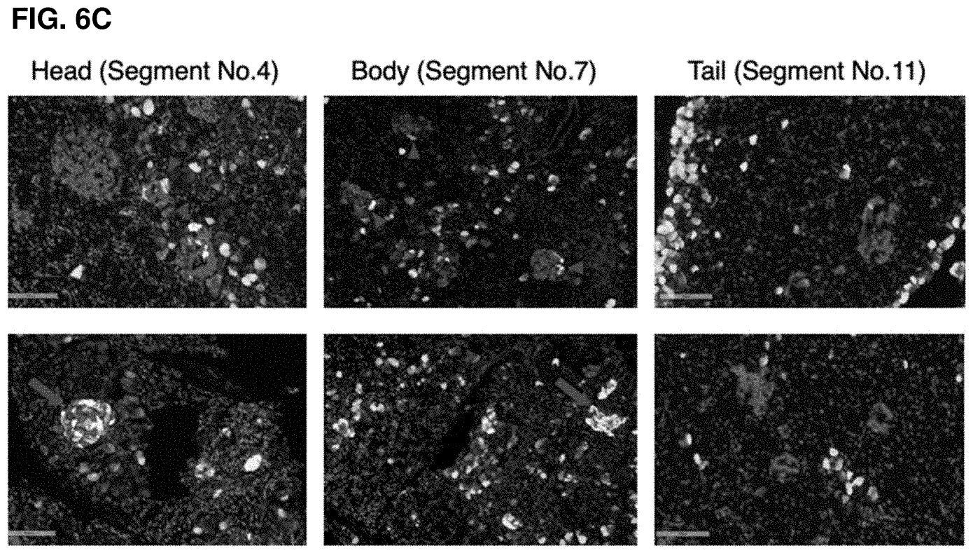

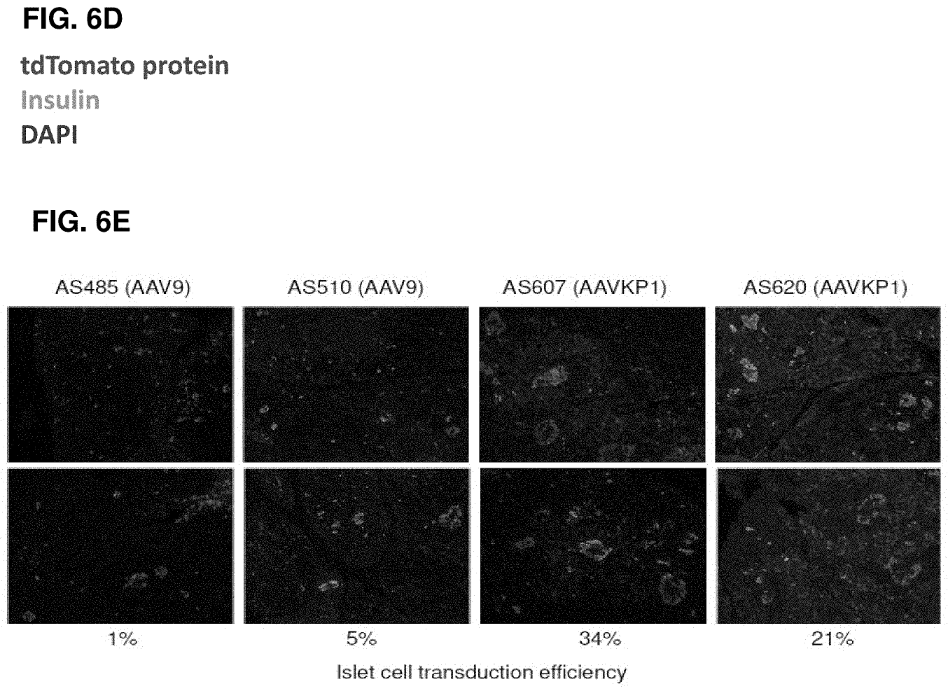

Two rhesus macaques were injected with AAV9-CAG-TdTomato via the PD at a dose of 1.4×10¹³ vg (Table 3) and four rhesus macaques were injected with AAV9-CAG-TdTomato via the PD at a dose of 8.4×10¹² to 1.4×10¹³ vg. Four weeks post-injection, various organs were harvested for histological and molecular analyses. The pancreas tissue was harvested before the animals were perfused with cold saline, split into 12 blocks (4 segments from each of the head, body and tail regions, FIG. 6A). Each segment of the pancreas tissue was immediately frozen or fixed with cold 4% paraformaldehyde or zinc formalin at room temperature. All the other organs were harvested after the completion of saline perfusion. Immunofluorescence microscopic analysis revealed many TdTomato-positive acinar cells and a much lesser number of TdTomato-positive endocrine cells in islets (FIG. 6C) in AAV9 vector-injected animals. Although many islets were negative for TdTomato expression, a small number of islets were effectively transduced showing vector-transduced cells in both the center and the periphery of islets (FIG. 6C, indicated with red arrows) and some islets were transduced only on the periphery (FIG. 6C, indicated with red arrowheads). The pancreatic duct transduction was not readily observed in the histology sections analyzed. In the AAVKP1 vector-injected animals, there were more AAV vector transduced cells. In particular, islet transduction efficiency was substantially higher in AAVKP1 vector-injected animals than AAV9 vector-injected animals (FIG. 6E). A semi-quantitative assessment of islet cell transduction efficiency revealed that 34% and 21% islet cells were transduced with AAVKP1 vector in the animals AS607 and AS620, respectively, while islet cell transduction efficiency with AAV9 was 1% and 5% in the animals AS485 and AS510, respectively. Transduced cells were not evenly distributed in the pancreatic tissues, and therefore the assessment of islet transduction efficiency was done only in the transduced areas.

Total DNA was also extracted from tissues that were harvested 4 weeks post-injection, and the vector genome copy numbers (vg per cell) were determined by qPCR (Table 7).

These observations demonstrate that retrograde intrapancreatic duct injection of AAV9 and AAVKP1 vector can mediate transduction in both acinar cells and islet cells with strong preference for acinar cells with AAV9 while there is a preference for islet cell transduction with AAVKP1. The endocrine cells in the center of islets are difficult to transduce with AAV9 compared to those on the periphery of islets and the islet cells are more effectively transduced with AAVKP1. With either of the AAV9 and AAVKP1, duct cell transduction was not observed. In mouse studies in which AAV6 and AAV8 and AAV9 vectors have been most commonly employed, it has been shown that, although AAV6 vectors can transduce pancreatic duct cells efficiently as well as acinar cells and islet cells following PD injection, duct cells are not susceptible to transduced with AAV8 or AAV9 vectors^{7,8}. This attribute of AAV9 showing resistance to transduction in duct cells is thus retained in non-human primates. In addition, preferential peripheral zone transduction in islets with intraductally delivered AAV9 vector in non-human primates is a reminiscence of the observation with intraductally delivered AAV6 and AAV8 vectors in mice highlighting a challenge to AAV vector-mediated gene delivery to the center of islets and uniform islet transduction following PD injection. These observations indicate that, although AAV9 vectors are capable of transducing acinar cells effectively following PD injection in non-human primates and potentially humans, effective islet transduction via the PD route for clinical translation will require more potent AAV capsids that can effectively penetrate into the center of islets. In this regard, AAVKP1 is an attractive AAV capsid for islet transduction via the PD route in the clinics.

### Example 6—Evaluation and Identification of AAV Capsids Efficiently Transducing Cells

To identify AAV capsids that can transduce pancreatic cells via the PD route in non-human primates better than AAV9 among the currently available AAV capsids including the common serotypes, new natural isolates, and the current state-of-the art capsids, we employed AAV DNA/RNA Barcode-Seq technology²⁻⁵. A DNA/RNA barcoded AAV library containing 36 AAV strains (Table 1) was produced and injected via the PD in two rhesus macaques at a dose of 8.5×10¹² vg. Various tissues were harvested 6 weeks post-injection for the AAV DNA/RNA Barcode-Seq analysis. The pancreas tissue was enzymatically digested, islet cells and non-islet cells were physically separated according to the standard protocol. Subsequently, each fraction was subjected to flow sorting using mouse monoclonal antibodies against endocrine cells (HIC1-2B4.2B), duct cells (DHIC5-4D9), or exocrine cells (HIC0-3B3). Briefly, endocrine cells were first separated from non-endocrine cells by co-labeling cells with HIC1-2B4.2B and DHIC5-4D9-PE, and duct cells and exocrine cells were then separated from non-labeled cells in the non-endocrine cell fraction. In this procedure, although a decent amount of pure endocrine cells and duct cells were obtained, only a small number of exocrine cells were recovered. This indicates that the mouse monoclonal antibody, HIC0-3B3, raised against human pancreatic exocrine cells, may not reliably label rhesus macaque exocrine cells. Thus, the AAV Barcode-Seq analysis was done using DNA and RNA extracted from the following three cell fractions flow-sorted from the pancreas: (1) endocrine cells, (2) duct cells and (3) other cells the majority of which should represent exocrine cells. The AAV Barcode-Seq analysis for other organs was performed using DNA extracted from bulk tissues. The results of the AAV Barcode-Seq analysis on vector transduction and biodistribution are summarized in Tables 4, 5, and 6. Only the data obtained from AS516 are shown. The AAV Barcode-Seq data obtained from AS532 was not informative because of the potential presence of anti-AAV antibodies against AAV9, the benchmark AAV serotype in this study. The observations support the conclusion that there are AAV capsids that are superior to AAV9 in pancreatic cell transduction following introduction into the pancreatic duct.

TABLE 4
Relative transduction efficiency of various AAV capsids in
pancreatic cell subpopulations

| | DNA Barcode-Seq | | | RNA Barcode-Seq | | |
|---|---|---|---|---|---|---|
| AAV Strain | Duct* | Islet** | Other*** | Duct | Islet | Other |
| AAV1 | 6.48 | 3.61 | 0.68 | 49.31 | 46.93 | 0.06 |
| AAV2 | 1.50 | 0.54 | 0.16 | 7.89 | 0.26 | 0.16 |
| AAV3 | 5.01 | 0.11 | 0.32 | 0.38 | 0.37 | 0.16 |
| AAV4 | 2.48 | 0.20 | 0.14 | 0.59 | 0.61 | 0.18 |
| AAV5 | 0.78 | 0.24 | 0.12 | 0.16 | 0.12 | 0.01 |
| AAV6 | 2.93 | 0.28 | 0.27 | 0.22 | 0.19 | 0.01 |
| AAV7 | 1.00 | 1.23 | 0.27 | 55.42 | 38.96 | 0.70 |
| AAV8 | 0.46 | 0.25 | 0.14 | 3.33 | 0.24 | 0.14 |
| AAV9 | 1.00 | 1.00 | 1.00 | 1.00 | 1.00 | 1.00 |
| AAV10 | 0.90 | 0.96 | 0.29 | 3.39 | 51.11 | 0.12 |
| AAV11 | 0.78 | 0.61 | 0.10 | 0.46 | 0.60 | 0.01 |
| AAVrh8 | 0.60 | 0.27 | 0.30 | 3.47 | 19.63 | 0.94 |
| AAVrh10 | 0.93 | 0.85 | 0.32 | 18.91 | 0.96 | 0.22 |
| AAVhu11 | 0.86 | 0.46 | 0.36 | 0.30 | 0.15 | 0.01 |
| AAVhu13 | 0.73 | 0.76 | 0.28 | 2.71 | 0.23 | 0.05 |
| AAVhu37 | 0.53 | 0.89 | 0.31 | 7.59 | 32.02 | 0.94 |
| AAVrh20 | 0.33 | 0.57 | 0.20 | 44.75 | 0.73 | 0.34 |
| AAVrh43 | 0.96 | 0.54 | 0.16 | 0.15 | 0.20 | 0.29 |
| AAVbb2 | 1.30 | 0.46 | 0.13 | 0.23 | 0.28 | 0.01 |
| AAVLK03 | 9.24 | 1.01 | 0.53 | 18.56 | 12.93 | 0.05 |
| AAVDJ | 2.33 | 6.07 | 0.59 | 10.30 | 137.83 | 1.02 |
| AAV2i8 | 0.62 | 1.53 | 0.42 | 0.31 | 34.97 | 0.02 |
| AAVR585E | 0.75 | 1.14 | 0.30 | 0.27 | 0.27 | 0.01 |
| AAV2G9 | 3.61 | 2.58 | 0.81 | 181.72 | 2.48 | 0.39 |
| AAVNP40 | 0.83 | 0.81 | 0.31 | 6.52 | 0.25 | 0.81 |
| AAVNP59 | 0.62 | 1.24 | 0.10 | 0.15 | 0.16 | 0.03 |
| AAVPHP.B | 0.70 | 0.51 | 0.09 | 0.33 | 1.18 | 0.78 |
| AAVPHP.eB | 0.53 | 0.41 | 0.08 | 1.04 | 33.78 | 0.17 |
| AAVPHP.S | 0.20 | 0.18 | 0.02 | 0.04 | 0.03 | 0.05 |
| AAVAnc80 | 0.34 | 1.46 | 0.08 | 1.07 | 27.30 | 0.01 |
| AAV2.7m8 | 2.30 | 1.65 | 0.23 | 0.39 | 88.45 | 0.02 |
| AAVsh10 | 26.68 | 2.71 | 1.98 | 51.92 | 49.11 | 0.06 |
| AAV2retro | 0.62 | 0.83 | 0.33 | 0.23 | 0.17 | 0.37 |
| AAVKP1 | 15.84 | 1.44 | 0.93 | 56.68 | 103.99 | 0.27 |
| AAVKP2 | 8.99 | 1.06 | 0.51 | 0.42 | 0.36 | 0.13 |
| AAVKP3 | 16.61 | 3.61 | 1.08 | 43.55 | 10.69 | 0.25 |

*Duct, duct cells.
**Islet, endocrine cells.
***Other, all other cells excluding duct cells and endocrine cells, presumably representing mainly exocrine cells (acinar cells).
Note:
Values and Phenotypic Difference values obtained by AAV Barcode-Seq analysis representing phenotypic differences compared to the phenotypes of AAV9, the benchmark capsids in the analysis (Adachi et al., Nature Communications 2014).

TABLE 5
Relative AAV vector genome transcriptional activity in the
pancreatic cells delivered by each different AAV capsid

| | AAV vector genome transcriptional activity | | |
|---|---|---|---|
| AAV Strain | Duct* | Islet** | Other*** |
| AAV1 | 7.61 | 13 | 0.09 |
| AAV2 | 5.24 | 0.47 | 1.01 |
| AAV3 | 0.08 | 3.32 | 0.51 |
| AAV4 | 0.24 | 3.03 | 1.28 |
| AAV5 | 0.21 | 0.48 | 0.05 |
| AAV6 | 0.08 | 0.68 | 0.04 |
| AAV7 | 55.55 | 31.67 | 2.56 |
| AAV8 | 7.25 | 0.94 | 0.99 |
| AAV9 | 1 | 1 | 1 |
| AAV10 | 3.76 | 53.21 | 0.4 |
| AAV11 | 0.59 | 0.98 | 0.12 |
| AAVrh8 | 5.78 | 73.82 | 3.18 |
| AAVrh10 | 20.31 | 1.12 | 0.69 |
| AAVhu11 | 0.35 | 0.32 | 0.04 |
| AAVhu13 | 3.73 | 0.31 | 0.18 |
| AAVhu37 | 14.31 | 36.01 | 3.08 |
| AAVrh20 | 134.08 | 1.28 | 1.69 |
| AAVrh43 | 0.16 | 0.36 | 1.82 |
| AAVbb2 | 0.18 | 0.61 | 0.09 |
| AAVLK03 | 2.01 | 12.76 | 0.09 |
| AAVDJ | 4.42 | 22.72 | 1.72 |
| AAV2i8 | 0.5 | 22.92 | 0.05 |
| AAVR585E | 0.37 | 0.23 | 0.04 |
| AAV2G9 | 50.4 | 0.96 | 0.48 |
| AAVNP40 | 7.84 | 0.3 | 2.61 |
| AAVNP59 | 0.24 | 0.13 | 0.26 |
| AAVPHP.B | 0.48 | 2.3 | 8.4 |
| AAVPHP.eB | 1.97 | 81.43 | 2.29 |
| AAVPHP.S | 0.17 | 0.18 | 2.75 |
| AAVAnc80 | 3.16 | 18.67 | 0.08 |
| AAV2.7m8 | 0.17 | 53.45 | 0.07 |
| AAVsh10 | 1.95 | 18.14 | 0.03 |
| AAV2retro | 0.37 | 0.21 | 1.13 |
| AAVKP1 | 3.58 | 72.37 | 0.29 |
| AAVKP2 | 0.05 | 0.34 | 0.25 |
| AAVKP3 | 2.62 | 2.96 | 0.23 |

*Duct, duct cells.
**Islet, endocrine cells.
***Other, all other cells excluding duct cells and endocrine cells, presumably representing mainly exocrine cells (acinar cells).
Note:
Values are determined by Phenotypic Difference value of RNA Barcode-Seq divided by Phenotypic Difference value of DNA Barcode Seq. The values shown in Table 4 are used.

TABLE 6
AAV vector genome biodistribution in off-
target organs following PD injection

| | DNA Barcode-Seq | | |
|---|---|---|---|
| AAV Strain | Heart | Liver | Kidney |
| AAV1 | 5.39 | 2.11 | 4.52 |
| AAV2 | 0.01 | 0.01 | 0.01 |
| AAV3 | 0.03 | 0.05 | 0.03 |
| AAV4 | 0.07 | 0.01 | 0.04 |
| AAV5 | 0.00 | 0.03 | 0.10 |
| AAV6 | 0.00 | 0.13 | 0.04 |
| AAV7 | 0.85 | 0.65 | 1.64 |
| AAV8 | 0.02 | 0.07 | 0.07 |
| AAV9 | 1.00 | 1.00 | 1.00 |
| AAV10 | 0.05 | 0.10 | 0.14 |
| AAV11 | 0.36 | 0.09 | 3.89 |
| AAVrh8 | 0.15 | 0.16 | 0.24 |
| AAVrh10 | 0.08 | 0.08 | 0.17 |
| AAVhu11 | 0.04 | 0.01 | 0.17 |
| AAVhu13 | 0.23 | 0.12 | 0.21 |
| AAVhu37 | 0.06 | 0.07 | 0.23 |
| AAVrh20 | 0.05 | 0.05 | 0.18 |
| AAVrh43 | 0.00 | 0.04 | 0.10 |
| AAVbb2 | 0.02 | 0.03 | 0.27 |
| AAVLK03 | 0.01 | 0.07 | 0.03 |
| AAVDJ | 0.28 | 0.07 | 0.06 |
| AAV2i8 | 0.15 | 0.04 | 0.13 |
| AAVR585E | 0.33 | 0.13 | 0.22 |
| AAV2G9 | 0.22 | 0.02 | 0.08 |
| AAVNP40 | 0.16 | 0.10 | 0.07 |
| AAVNP59 | 0.04 | 0.25 | 0.00 |
| AAVPHP.B | 0.19 | 0.48 | 0.28 |
| AAVPHP.eB | 0.10 | 0.17 | 0.33 |
| AAVPHP.S | 0.01 | 0.01 | 0.04 |
| AAVAnc80 | 0.01 | 0.02 | 0.01 |
| AAV2.7m8 | 0.01 | 0.00 | 0.04 |
| AAVsh10 | 0.09 | 0.44 | 0.03 |
| AAV2retro | 0.07 | 0.03 | 0.07 |
| AAVKP1 | 0.10 | 0.02 | 0.03 |
| AAVKP2 | 0.09 | 0.01 | 0.01 |
| AAVKP3 | 0.09 | 0.02 | 0.05 |

Note:
Values and Phenotypic Difference values obtained by our AAV Barcode-Seq analysis representing phenotypic differences compared to the phenotypes of AAV9, the benchmark capsids in the analysis (Adachi et al., Nature Communications 2014).

The results achieved are summarized below.

(1) The following AAV capsids can transduce pancreatic endocrine cells more than 10× better than AAV9 following PD injection: AAVDJ, AAVKP1, AAV2.7m8, AAV10, AAVsh10, AAV1, AAV7, AAV2i8, AAVPHPeB, AAVhu37, AAVAnc80, AAVrh8, AAVLK03, and AAVKP3. In particular, AAVDJ, AAVKP1, AAV2.7m8, AAV10, AAVsh10, and AAV1 are highly efficient, transducing endocrine cells 134×, 104×, 88×, 51×, 49×, 47×, and 41× more efficient than AAV9, respectively.

(2) The following AAV capsids can transduce pancreatic endocrine cells up to 10× better than AAV9 following PD injection: AAV2G9, and AAVPHPB.

(3) The following AAV capsids can transduce pancreatic duct cells more than 10× better than AAV9 following PD injection: AAV2G9, AAVKP1, AAV7, AAVsh10, AAV1, AAVrh20, AAVKP3, AAVrh10, AAVLK03, and AAVDJ. In particular, AAV2G9, AAVKP1, AAV7, AAVsh10, AAV1, AAVrh20, and AAVKP3 are highly efficient, transducing pancreatic duct cells 182×, 57×, 55×, 52×, 49×, 44×, and 44× more efficient than AAV9, respectively.

(4) The following AAV capsids can transduce pancreatic duct cells up to 10× better than AAV9 following PD injection: AAV2, AAVhu37, AAVNP40, AAVrh8, AAV10, AAV8, AAVhu13, AAVAnc80, and AAVPHPeB.

(5) The following AAV capsids can transduce pancreatic exocrine cells nearly equivalent (0.7× to 1.0×) to AAV9: AAVDJ, AAVhu37, AAVrh8, AAVNP40, AAVPHPB, and AAV7. Among them, AAVDJ, AAVhu37, AAVrh8 and AAVNP40 show <0.3× off-target transduction efficiency compared to AAV9

(6) The following AAV capsids can transduce both pancreatic endocrine cells and duct cells more than 10× better than AAV9 following PD injection: AAVKP1, AAV7, AAVsh10, AAV1, AAVKP3, AAVLK03, and AAVDJ. In particular, AAVKP1, AAV7, AAVsh10, and AAV1 are highly efficient, transducing both endocrine cells and duct cells >38× more efficient than AAV9.

(7) The following AAV capsids can transduce pancreatic endocrine cells more than 10× better than AAV9 and detarget duct cells and exocrine cells, showing high specificity to endocrine cells: AAV2i8 and AAV2.7m8.

(8) The following AAV capsids can transduce pancreatic endocrine cells more than 10× better than AAV9 while transduction enhancement in duct cells and exocrine cells is not observed or is minimum (up to 4×), showing relative specificity to endocrine cells: AAVPHPeB, AAVAnc80, AAV10, and AAVrh8.

(9) The following AAV capsids can transduce pancreatic duct cells more than 10× better than AAV9 and detarget endocrine cells and exocrine cells, showing specificity to duct cells: AAVrh20.

(10) The following AAV capsids can transduce pancreatic duct cells more than 10× better than AAV9 while transduction enhancement in endocrine cells and exocrine cells is not observed or is minimum (up to 3×), showing relative specificity to duct cells: AAV2G9 and AAVrh10.

(11) The following AAV capsids detarget pancreatic endocrine cells resulting in <=0.2× endocrine cells transduction compared to AAV9: AAVPHPS, AAV5, AAVhu11, AAVNP59, AAV2retro, AAV6, and AAVrh43. Their efficiencies of endocrine cells transduction compared to the transduction with AAV9 are 0.03×, 0.12×, 0.12×, 0.13×, 0.13×, 0.15×, 0.16×, 0.17×, 0.19×, and 0.20×, respectively.

(12) The following AAV capsids detarget pancreatic duct cells resulting in <=0.2× duct cells transduction compared to AAV9: AAVPHPS, AAVNP59, AAVrh43, and AAV5. Their efficiencies of endocrine cells transduction compared to the transduction with AAV9 are 0.04×, 0.10×, 0.10×, 0.13×, 0.15×, 0.15×, 0.16×, and 0.16×, respectively.

(13) The following AAV capsids detarget pancreatic exocrine cells resulting in <=0.2× exocrine cells transduction compared to AAV9: AAV5,

AAVhu11, AAV6, AAVR585E, AAVbb2, AAV11, AAVAnc80, AAV2i8, AAV2.7m8, AAVNP59, AAVPHPS, AAVhu13, AAVLK03, AAV1, AAVsh10, AAV10, AAVKP2, AAV8, AAV2, AAV3, AAVPHPeB, and AAV4. Their efficiencies of endocrine cells transduction compared to the transduction with AAV9 are 0.00×, 0.01×, 0.01×, 0.01×, 0.01×, 0.01×, 0.01×, 0.01×, 0.01×, 0.01×, 0.01×, 0.01×, 0.02×, 0.02×, 0.03×, 0.03×, 0.05×, 0.05×, 0.05×, 0.05×, 0.05×, 0.06×, 0.06×, 0.12×, 0.13×, 0.14×, 0.16×, 0.16×, 0.17×, 0.18×, and 0.19×, respectively.

(14) The following AAV capsids detarget pancreatic endocrine cells, duct cells and exocrine cells resulting in <=0.2× transduction in all three cell populations compared to AAV9: AAV5, AAVNP59, and AAVPHPS.

(15) AAV vector genome DNA delivered by the following AAV capsids show transcriptional activities that are >10× higher than AAV9 vector genome transcriptional activities in pancreatic endocrine cells following PD injection: AAVPHPeB, AAVrh8, AAVKP1, AAV2.7m8, AAV10, AAVhu37, AAV7, AAV2i8, AAVDJ, AAVAnc80, AAVsh10, AAV1, and AAVLK03. In particular, the vector genome DNA delivered by AAVPHPeB, AAVrh8, AAVKP1, AAV2.7m8, and AAV10 show 81×, 74×, 72×, 53×, and 53× higher transcriptional activities than those delivered by AAV9.

(16) AAV vector genome DNA delivered by the following AAV capsids show transcriptional activities that are >10× higher than AAV9 vector genome transcriptional activities in pancreatic duct cells following PD injection: AAVrh20, AAV7, AAV2G9, AAVrh10, and AAVhu37. In particular, the vector genome DNA delivered by AAVrh20, AAV7, and AAV2G9 show 138×, 56×and 50× higher transcriptional activities than those delivered by AAV9.

(17) The following AAV capsids detarget the liver resulting in <=0.2× vector genome DNA copy numbers in the liver compared to AAV9: AAV2.7m8, AAV2, AAV4, AAVhu11, AAVPHPS, AAVKP2, AAV2G9, AAVAnc80, AAVKP1, AAVKP3, AAVS, AAVbb2, AAV2retro, AAVrh43, AAV2i8, AAV3, AAVrh20, AAV8, AAVhu37, AAVLK03, AAVDJ, AAVrh10, AAV11, AAV10, AAVNP40, AAVhu13, AAV6, AAVR585E, AAVrh8, and AAVPHPeB. Their vector genome copy numbers in the liver compared to the AAV9 vector genome copy number are 0.00×, 0.00×, 0.00×, 0.00×, 0.01×, 0.01×, 0.01×, 0.01×, 0.01×, 0.01×, 0.02×, 0.02×, 0.02×, 0.02×, 0.03×, 0.03×, 0.03×, 0.04×, 0.04×, 0.05×, 0.05×, 0.06×, 0.07×, 0.07×, 0.07×, 0.07×, 0.07×, 0.08×, 0.09×, 0.10×, 0.10×, 0.11×, 0.12×, 0.13×, 0.13×, 0.13×, 0.16×, and 0.17×, respectively.

(18) The following AAV capsids detarget the liver, heart and kidney resulting in <=0.2× vector genome DNA copy numbers in these organs compared to AAV9: AAV2, AAVAnc80, AAVKP2, AAVLK03, AAV3, AAVKP1, AAV6, AAV2.7m8, AAVPHPS, AAV4, AAVKP3, AAV8, AAV2retro, AAVNP40, AAVS, AAVrh43, AAV2i8, AAV10, AAVhu11, AAVrh10, and AAVrh20. In particular, AAV2, AAVAnc80, AAVKP2, AAVLK03, AAV3, AAVKP1, AAV2.7m8, AAVPHPS, AAV4, AAVKP3, AAV8, AAV2retro, AAVS, and AAVrh43 show <=0.1× genome dissemination to these organs compared to AAV9; AAV2.7m8, AAV2, AAVPHPS, AAVAnc80, and AAV3 show <=0.05× genome dissemination to these organs compared to AAV9.

It should be noted that AAV6 and AAV8 capsids, which have been most extensively used in rodent studies as effective AAV capsids, are not all ideal for pancreatic transduction in primates; therefore, they have minimal clinical relevance in AAV vector-mediated gene delivery to the pancreas via the PD route.

Pancreatic cell transduction with AAVKP1 vector following PD injection. As described above, the AAV Barcode-Seq analysis revealed that AAVDJ and AAVKP1 can transduce pancreatic islet cells >100× better than AAV9. Based on the observation by Pekrun et al. that AAVKP1 transduces human isles and hESC-derived β-cells more efficiently than AAVDJ, AAVKP1 was selected for the subsequent validation study using rhesus macaques. A rhesus macaque (AS567, 3.3-year-old male, 5.3 kg, see Table 3) was injected with AAVKP1-CAG-TdTomato at a dose of 8.4×10¹² vg using a method that was modified to reduce intrapancreatic pressure during the PD injection as described herein (e.g., aspiration of pancreatic juice in the PD, Octreotide injection, injection of an AAV vector agent from the tail using a microcatheter, and reduced agent dwelling time). Among the 7 animals that underwent the PD injection procedure, only this animal showed no acinarization pattern on pancreatography. Four weeks after injection, the pancreas tissue was processed in the same way as that for other animals and AAV vector-mediated transduction in the pancreas was assessed by immunofluorescence microscopy. The histological assessment was performed on the pancreatic segments Nos. 3, 4, 7, 8, 11 and 12 (see FIG. 6A) but only very few TdTomato positive cells were seen. Then by quantitative PCR, AAV vector genome DNA copy numbers in the pancreatic segments Nos. 2, 6 and 10 (see FIG. 6A), liver, heart, kidney and duodenum were quantified. The result demonstrated that, unlike AAV9 vector-treated animals showing relatively even vector genome distribution across the head, body and tail regions in the pancreas, all of the AAVKP1-treated animals showed a remarkably uneven vector genome distribution pattern (Table 8). In AS567, only the segment No.10 in the tail contained a decent number of vector genomes, which corroborates the poor transduction in the pancreatic head and body regions demonstrated by immunofluorescence microscopy. A reason may be that the microcatheter tip was placed in the segment No.10 and vector solution did not reach the segments Nos. 11 and 12 by the injection method we used for this animal, resulting in transduction only in the segment No.10. Although the histological assessment of transduction efficiency in the pancreatic tail was performed only for the segments No. 11 and No. 12, showing poor transduction, it is possible that the segment No.10 could show much higher vector transduction than that attainable by AAV9, particularly in islets, considering that this segment has a 3× higher AAVKP1 vector genome copy number (Table 8) and AAVKP1 vector genome DNA transcriptional activity has been found to be 73× higher than that of AAV9 (Table 5). Thus, these observations strongly indicate that PD injection of AAVKP1 mediates much higher pancreatic cell transduction in non-human primates than AAV9.

TABLE 8
AAV vector genome copy numbers

| | | AAV vector genome copy number (vg/dge*) | | | | | | |
|---|---|---|---|---|---|---|---|---|
| | | Pancreas** | | | | | | |
| | | Head | Body | Tail | | | | |
| Animal | | (Seg. | (Seg. | (Seg. | | | Kid- | Duo- |
| ID | Vector | No. 2) | No. 6) | No. 10) | Liver | Heart | ney | denum |
| AS485 | AAV9 | 2.6 | 6.1 | 4.8 | 14.8 | 0.3 | 0.6 | 0.7 |
| AS510 | AAV9 | 2.0 | 3.6 | 5.5 | 8.0 | 1.1 | 1.6 | 18.1 |
| AS567 | AAVKP1 | 0.1 | 0.1 | 14.4 | 0.1 | 0.0 | 0.0 | 0.0 |
| AS606 | AAVKP1 | 0.7 | 0.8 | 2.9 | 0.0 | 0.0 | 0.0 | 0.0 |
| AS607 | AAVKP1 | 4.9 | 5.7 | 35.3 | 0.1 | 0.1 | 0.0 | 0.1 |
| AS620 | AAVKP1 | 2.5 | 5.5 | 26.1 | 0.1 | 0.0 | 0.0 | 0.0 |

*vg/dge, vector genomes per diploid genomic equivalent
**For the segment information, please refer to FIG. 6A.

### Example 7—Evaluation of Pharmacokinetic Profile of AAV Vectors

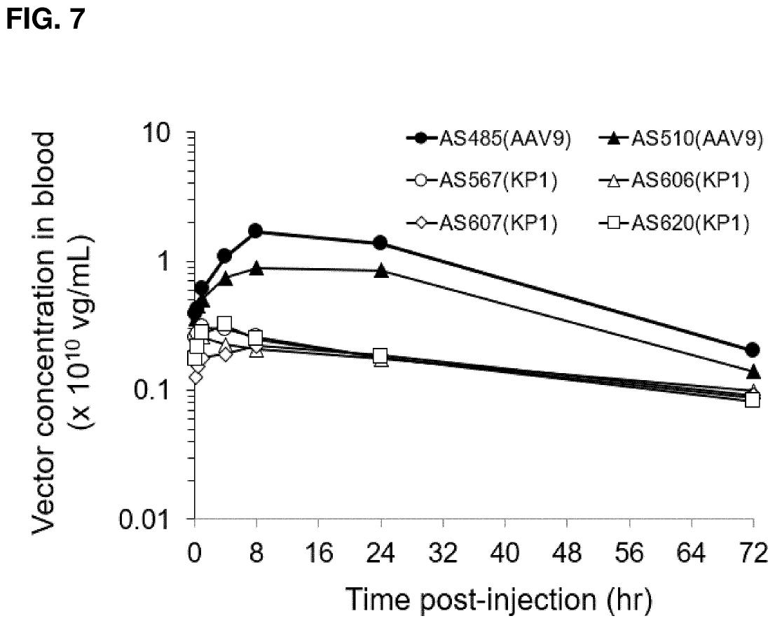

During the course of the animal experiments, blood samples were also collected at multiple time points up to 72 hours post-injection for a pharmacokinetic analysis as done previously². Blood samples were collected at 10, 30 min, 1, 4, 8, 24 and 72 hours post-injection. Using the samples collected from the si×animals that were injected with AAV9 vector (AS485 and AS510) or AAVKP1 vector (AS567, AS606, AS607 and AS620), AAV vector concentrations were determined in the blood by quantitative PCR (FIG. 7). The AAV9 blood vector concentrations peaked 8 hours post-injection while AAVKP1 showed much lower vector concentrations and an earlier peak following PD injection. Using the samples collected from one animal injected with the AAV barcode library, relative vector genome concentrations of each AAV capsid in the blood compared to the concentration of AAV9 by AAV Barcode-Seq (Table 9) were determined.

TABLE 9
Pharmacokinetic profiles of various AAV capsids delivered
by PD injection in a non-human primate.

| | Time after PD injection | | | | | | |
|---|---|---|---|---|---|---|---|
| AAV Strain | 10 min | 30 min | 1 h | 4 h | 8 h | 24 h | 72 h |
| AAV1 | 0.65 | 0.86 | 0.92 | 0.56 | 0.39 | 0.08 | 1.12 |
| AAV2 | 0.10 | 0.11 | 0.10 | 0.09 | 0.07 | 0.03 | 0.06 |
| AAV3 | 0.03 | 0.01 | 0.01 | 0.00 | 0.00 | 0.00 | 0.04 |
| AAV4 | 0.06 | 0.02 | 0.01 | 0.01 | 0.01 | 0.01 | 0.30 |
| AAV5 | 0.18 | 0.15 | 0.08 | 0.05 | 0.05 | 0.03 | 1.18 |
| AAV6 | 0.06 | 0.08 | 0.06 | 0.01 | 0.01 | 0.00 | 0.02 |
| AAV7 | 0.91 | 0.98 | 1.05 | 0.98 | 0.97 | 0.80 | 0.30 |
| AAV8 | 0.63 | 0.52 | 0.43 | 0.27 | 0.12 | 0.01 | 0.02 |
| AAV9 | 1.00 | 1.00 | 1.00 | 1.00 | 1.00 | 1.00 | 1.00 |
| AAV10 | 1.06 | 0.91 | 0.74 | 0.28 | 0.30 | 0.08 | 0.23 |
| AAV11 | 0.36 | 0.52 | 0.50 | 0.40 | 0.23 | 0.01 | 1.28 |
| AAVrh8 | 1.19 | 1.03 | 0.89 | 0.54 | 0.58 | 0.24 | 0.09 |
| AAVrh10 | 1.05 | 0.89 | 0.72 | 0.26 | 0.28 | 0.07 | 0.20 |
| AAVhu11 | 0.69 | 0.58 | 0.49 | 0.37 | 0.33 | 0.36 | 0.20 |
| AAVhu13 | 0.66 | 0.64 | 0.45 | 0.41 | 0.41 | 0.45 | 0.36 |
| AAVhu37 | 1.17 | 1.06 | 0.86 | 0.33 | 0.34 | 0.08 | 0.22 |
| AAVrh20 | 1.01 | 0.93 | 0.76 | 0.27 | 0.27 | 0.07 | 0.15 |
| AAVrh43 | 0.64 | 0.47 | 0.42 | 0.30 | 0.11 | 0.01 | 0.00 |
| AAVbb2 | 1.03 | 1.05 | 0.88 | 0.44 | 0.46 | 0.14 | 0.09 |
| AAVLK03 | 0.03 | 0.01 | 0.00 | 0.00 | 0.00 | 0.00 | 0.02 |
| AAVDJ | 0.22 | 0.12 | 0.11 | 0.05 | 0.03 | 0.01 | 0.03 |
| AAV2i8 | 0.67 | 0.58 | 0.46 | 0.45 | 0.47 | 0.54 | 0.55 |
| AAVR585E | 0.64 | 0.57 | 0.47 | 0.40 | 0.40 | 0.42 | 0.27 |
| AAV2G9 | 0.09 | 0.10 | 0.08 | 0.05 | 0.06 | 0.04 | 0.01 |
| AAVNP40 | 0.58 | 0.45 | 0.34 | 0.18 | 0.22 | 0.19 | 0.09 |
| AAVNP59 | 0.38 | 0.16 | 0.09 | 0.03 | 0.02 | 0.01 | 0.02 |
| AAVPHP.B | 0.82 | 0.87 | 0.88 | 0.90 | 0.74 | 0.19 | 0.12 |
| AAVPHP.eB | 0.95 | 1.13 | 1.17 | 1.35 | 1.26 | 0.47 | 0.06 |
| AAVPHP.S | 0.07 | 0.05 | 0.03 | 0.03 | 0.04 | 0.05 | 0.01 |
| AAVAnc80 | 0.34 | 0.33 | 0.23 | 0.08 | 0.03 | 0.01 | 0.00 |
| AAV2.7m8 | 0.11 | 0.08 | 0.07 | 0.05 | 0.04 | 0.02 | 0.00 |
| AAVsh10 | 0.12 | 0.03 | 0.01 | 0.00 | 0.00 | 0.00 | 0.21 |
| AAV2retro | 0.55 | 0.54 | 0.44 | 0.16 | 0.11 | 0.05 | 0.01 |
| AAVKP1 | 0.04 | 0.01 | 0.00 | 0.00 | 0.00 | 0.00 | 0.00 |
| AAVKP2 | 0.03 | 0.01 | 0.01 | 0.00 | 0.00 | 0.00 | 0.01 |
| AAVKP3 | 0.03 | 0.01 | 0.01 | 0.00 | 0.00 | 0.00 | 0.00 |

Note:
Values and Phenotypic Difference values obtained by our AAV Barcode-Seq analysis representing phenotypic differences compared to the phenotypes of AAV9, the benchmark capsids in the analysis (Adachi et al., Nature Communications 2014).

The key discoveries made in the pharmacokinetic analysis can be summarized as follows.

(1) The following AAV capsids leak out of the pancreas and enter the blood circulation <=0.2× efficiently than AAV9, which can be assessed by the blood concentrations 10 min post-PD injection: AAV3, AAVLK03, AAVKP2, AAVKP3, AAVKP1, AAV4, AAV6, AAVPHPS, AAV2G9, AAV2, AAV2.7m8, AAVsh10, and AAV5. Their relative blood concentrations 10 min post-injection compared to the AAV9 vector concentration are 0.03×, 0.03×, 0.03×, 0.03×, 0.04×, 0.06×, 0.06×, 0.07×, 0.09×, 0.10×, 0.11×, 0.12×, and 0.18×, respectively.

(2) The following AAV capsids leak out of the pancreas, enter the blood circulation, and maintain their blood concentrations throughout the 72 hour-long period following PD injection <=0.2× efficiently than AAV9, which exhibit the most attractive pharmacokinetic profile minimizing off-target vector genome dissemination: AAVKP3, AAVKP2, AAVLK03, AAV3, AAVKP1, AAV6, AAVPHPS, AAV2G9, AAV2, and AAV2.7m8.

(3) The following AAV capsids leak out of the pancreas and enter the blood circulation as efficiently as AAV9 (>=0.8× compared to AAV9), which can be assessed by the blood concentrations 10 min post-injection: AAVPHPB, AAV7, AAVPHPeB, AAVrh20, AAVbb2, AAVrh10, AAV10, AAVhu37, and AAVrh8. Their relative blood concentrations 10 min post-injection compared to the AAV9 vector concentration are 0.82×, 0.82×, 0.85×, 0.86×, 0.86×, 0.88×, 0.91×, 0.95×, 0.98×, 1.01×, 1.03×, 1.03×, 1.03×, 1.05×, 1.06×, 1.17×, and 1.19, respectively.

(4) The following AAV capsids maintain blood concentrations as high as AAV9 (>=0.8× compared to AAV9 at any of the time points): AAVPHPB, AAVrh20, AAVbb2, AAVrh10, AAV7, AAV10, AAV1, AAVhu37, AAV5, AAVrh8, AAV11, and AAVPHPeB. Their maximum relative blood concentrations compared to the AAV9 vector concentration are 0.82×, 0.87×, 0.90×, 0.97×, 1.01×, 1.01×, 1.02×, 1.05×, 1.05×, 1.05×, 1.06×, 1.12×, 1.14×, 1.17×, 1.18×, 1.19×, 1.28×, 1.35×, 1.97×, and 2.11×.

### Example 8—Testing Safety of Intraductal Introduction of AAV Vectors

All the animals that underwent the surgical procedure described herein survived without any major complications until the end of the experiments. The blood test results including serum amylase and lipase levels in six animals are shown in Tables 10, 11, 12, 13, and 14. The data for the remaining 3 animals are not included in the tables, but showed the same profiles. No remarkable abnormalities in the blood tests were observed except for transient elevation of serum amylase and lipase levels in all the treated animals. Although mild loss of appetite was observed in some animals, clinical signs suggesting post-procedure pancreatitis or peritonitis have developed in none of the treated animals, and the amylase and lipase levels went down to the normal range within 3 days following the procedure in all the treated animals. These observations demonstrate that injection of 1.0 to 2.0 ml of AAV vector-containing solution over 2 min into the PD followed by outflow blockage to attain a 20-min dwelling time in 2.2-to-9.8 kg rhesus macaques (the median body weight, 3.7 kg; the average body weight, 4.2 kg) is safe. This volume can be translated to approximately 10 to 15 ml for young adult and adult human subjects. The injection volume and rate significantly affect AAV vector transduction efficiency, and in mice, approximately 100 μL is the standard volume for PD injection for effective islet cell transduction⁹. If the same injection volume were to be required for an adult human subject weighing 60 kb (132 lb.), a volume of 300 mL of AAV vector solution would be injected into the PD in the human pancreas, which should not be feasible. Histological assessment of hematoxylin-eosin (HE)-stained pancreatic tissues revealed an modest increase in the number of infiltrating lymphocytes and some fibrosis, which we presume to be primarily caused by host-immune response against an immunogenic transgene (i.e., TdTomato) and not to be due primarily to the procedure-induced tissue damages. In this regard, the observations obtained from the disclosed non-human primate study indicates that, with the appropriately optimized PD injection procedure disclosed herein, very effective pancreatic cell transduction will be safely attainable with a volume that is much smaller than that has been required for rodent studies.

TABLE 10
White blood cell counts before and after PD injection (cells/μL)

| | Animal ID | | | | | |
|---|---|---|---|---|---|---|
| Time point | AS485 | AS510 | AS516 | AS532 | AS567 | AS606 |
| Pre | 6000 | 9800 | 5800 | 6800 | 7100 | 8800 |
| 4 h | | | | | | |
| 24 h | | | | | | |
| 3 d | 6100 | 7900 | 4100 | 8500 | 6100 | 6200 |
| 7 d | 7100 | 11300 | 9900 | 8700 | 7700 | n.d. |

Normal range, 4200 to 13600.

TABLE 11
Serum alanine aminotransferase (ALT) levels
before and after PD injection (U/L)

| | Animal ID | | | | | |
|---|---|---|---|---|---|---|
| Time point | AS485 | AS510 | AS516 | AS532 | AS567 | AS606 |
| Pre | 47 | 30 | 30 | 26 | 27 | 27 |
| 4 h | | 57 | 45 | 29 | 40 | 80 |
| 24 h | 81 | 119 | 90 | 69 | 67 | 104 |
| 3 d | 51 | 57 | 63 | 52 | 43 | 94 |
| 7 d | 45 | 30 | 37 | 38 | 32 | 46* |

Normal range, 19 to 94.
*measured 5 d post-injection.

TABLE 12
Serum glucose levels before and after PD injection (mg/dL)

| | Animal ID | | | | | |
|---|---|---|---|---|---|---|
| Time point | AS485 | AS510 | AS516 | AS532 | AS567 | AS606 |
| Pre | 58 | 70 | 87 | 85 | 87 | 40 |
| 4 h | 69 | 71 | 79 | 73 | 123 | 80 |
| 24 h | 82 | 91 | 86 | 85 | 95 | 84 |
| 3 d | | 33 | 62 | 90 | 63 | 61 |
| 7 d | 89 | 60 | 60 | 60 | 86 | 51* |

Normal range, 45 to 93.
*measured 5 d post-injection.

TABLE 13
Serum glucose levels before and after PD injection (mg/dL)

| | Animal ID | | | | | |
|---|---|---|---|---|---|---|
| Time point | AS485 | AS510 | AS516 | AS532 | AS567 | AS606 |
| Pre | 343 | 416 | 348 | 419 | 335 | 570 |
| 4 h | 730 | 1742 | 3563 | 1360 | 2551 | 1950 |
| 24 h | 1281 | 2405 | 1597 | 1186 | 3429 | 4192 |
| 3 d | 394 | 653 | 408 | 625 | 336 | 581 |
| 7 d | 358 | 743 | 423 | 398 | 270 | 586* |

Normal range, 206 to 844.
*measured 5 d post-injection.

TABLE 14
Serum lipase levels before and after PD injection (U/L)

| | Animal ID | | | | | |
|---|---|---|---|---|---|---|
| Time point | AS485 | AS510 | AS516 | AS532 | AS567 | AS606 |
| Pre | 17 | 23 | 16 | 27 | 31 | 46 |
| 4 h | 98 | 577 | 802 | 414 | 541 | 477 |
| 24 h | 19 | 658 | 255 | 121 | 766 | 772 |
| 3 d | 16 | 27 | 15 | 13 | 14 | 19 |
| 7 d | | 32 | | 17 | 26 | 50 |

Normal range, 10 to 140.

References (the contents of which are incorporated herein by reference)

1. Pekrun, K. et al. JCI Insight 4, (2019), PMCID:PMC6948855, PMID:31723052.

2. Adachi, K. et al. Nat Commun 5, 3075, (2014), PMCID:PMC3941020, PMID:24435020.

3. Adachi, K., Nakai, H. International Patent Application Published Under the PCT. WO 2017/192750 Al

(https://patents.google.com/patent/WO2018119330A2/en), (2017).

4. Adachi, K., Nakai, H. International Patent Application Published Under the PCT. WO 2018/119330A2

(https://patents.google.com/patent/WO2018119330A2/en), (2018).

5. Adachi, K. et al. Molecular therapy : the journal of the American Society of Gene Therapy 22, (2014).

6. Adachi, K. et al. Sci Rep 10, 10073, (2020), PMCID:PMC7308316, PMID:32572045.

7. Jimenez, V. et al. Diabetologia 54, 1075-1086, (2011), PMID:21311856.

8. Quirin, K. A. et al. Mol Ther Methods Clin Dev 8, 8-20, (2018), PMCID:PMC5675991, PMID:29349096.

9. Xiao, X. et al. Nat Protoc 9, 2719-2724, (2014), PMCID:PMC4734891, PMID:25356582.

It will be apparent to those having skill in the art that many changes may be made to the details of the above-described embodiments without departing from the underlying principles of the invention. The scope of the present invention should, therefore, be determined only by the following claims.

1. A method of delivering a composition comprising an adeno-associated virus (AAV) to one or more pancreatic cell types in a subject, comprising:
a. guiding a distal end of a catheter to a major duodenal papilla in a duodenum of the subject;
b. advancing the distal end of the catheter in a retrograde direction through the pancreatic duct to a delivery point internal to the pancreas of the subject;
c. delivering a therapeutically effective amount of the composition through the catheter to the delivery point; and
d. allowing the composition to reside at the delivery point for a dwell time,
wherein the distal end of the catheter has a diameter selected to inhibit backflow of the AAV, and
wherein the AAV comprises a genome and a capsid protein comprising the amino acid sequence of SEQ ID NO: 1, SEQ ID NO: 2, SEQ ID NO: 7, SEQ ID NO:
8, SEQ ID NO: 10, SEQ ID NO: 12, SEQ ID NO: 13, SEQ ID NO: 15, SEQ ID NO:
16, SEQ ID NO: 17, SEQ ID NO: 20, SEQ ID NO: 21, SEQ ID NO: 22, SEQ ID NO:
24, SEQ ID NO: 25, SEQ ID NO: 27, SEQ ID NO: 28, SEQ ID NO: 33, SEQ ID NO:
34, SEQ ID NO: 35, SEQ ID NO: 39, or SEQ ID NO: 41, or an amino acid sequence at least 95% identical thereto,
thereby delivering the composition comprising the AAV to the one or more pancreatic cell types in the subject.

2. The method of claim 1, wherein the delivery point is located:
a) distal to the head of the pancreas;
b) in the body of the pancreas; or
c) in the tail of the pancreas.

3. The method of claim 1, wherein the dwell time is:
a) about 5 minutes to about 30 minutes; or
b) about 10 minutes to about 20 minutes.

4. The method of claim 1, wherein the guiding comprises accessing the duodenum via endoscopy.

5. The method of claim 4, wherein the guiding step further comprises duodenotomy.

6. The method of claim 1, wherein the AAV transduces at least one of islet cells, duct cells, and acinar cells.

7. The method of claim 1, wherein the one or more pancreatic cell types comprise islet cells, and the capsid protein comprises the amino acid sequence of SEQ ID NO: 1, SEQ ID NO: 7, SEQ ID NO: 10, SEQ ID NO: 12, SEQ ID NO: 16, SEQ ID NO: 20, SEQ ID NO: 21, SEQ ID NO: 22, SEQ ID NO: 24, SEQ ID NO: 27, SEQ ID NO: 28, SEQ ID NO: 33, SEQ ID NO: 34, SEQ ID NO: 35, SEQ ID NO: 39, SEQ ID NO: 41 or an amino acid sequence at least 95% identical thereto.

8. The method of claim 6, wherein the one or more pancreatic cell types comprise duct cells and the capsid protein comprises the amino acid sequence of SEQ ID NO: 1, SEQ ID NO: 2, SEQ ID NO: 7, SEQ ID NO: 8, SEQ ID NO: 10, SEQ ID NO: 12, SEQ ID NO: 13, SEQ ID NO: 15, SEQ ID NO: 16, SEQ ID NO: 17, SEQ ID NO: 20, SEQ ID NO: 21, SEQ ID NO: 24, SEQ ID NO: 25, SEQ ID NO: 28, SEQ ID NO: 33, SEQ ID NO: 35, SEQ ID NO: 39, SEQ ID NO: 41 or an amino acid sequence at least 95% identical thereto.

9. The method of claim 6, wherein the one or more pancreatic cell types comprise acinar cells and the capsid protein comprises the amino acid sequence of SEQ ID NO: 7, SEQ ID NO: 12, SEQ ID NO: 16, SEQ ID NO: 21, SEQ ID NO: 25, SEQ ID NO: 27, or an amino acid sequence at least 95% identical thereto.

10. The method of claim 1, wherein the AAV transduces the one or more pancreatic cell types with an efficiency about 5 times to about 200 times greater than that of AAV9.

11. The method of claim 1, wherein the AAV transduces the one or more pancreatic cell types with an efficiency about 40 times to about 200 times greater than that of AAV9.

12. The method of claim 1, wherein the AAV vector detargets a pancreatic cell type, resulting in transduction of the pancreatic cell type less than about 0.25 times that of AAV9.

13. The method of claim 1, wherein the AAV vector disseminates to liver, heart, and/or kidney, at a level less than about 0.1 times that of AAV9.

14. The method of claim 1, wherein the genome comprises a nucleic acid molecule encoding a therapeutic protein.

15. The method of claim 1, further comprising controlling a fluid pressure within the pancreatic duct to reduce incidence of pancreatitis.

16. The method of claim 1, comprising aspirating an amount of pancreatic fluid from the pancreatic duct before delivering the AAV.

17. A method of treating a pancreatic disorder in a subject, comprising:
performing endoscopic retrograde cholangiopancreatography to transduce pancreatic cells of the patient with an AAV comprising a genome and a capsid protein comprising the amino acid sequence of SEQ ID NO: 1, SEQ ID NO: 7, SEQ ID NO: 10, SEQ ID NO: 12, SEQ ID NO: 16, SEQ ID NO: 20, SEQ ID NO: 21, SEQ ID NO: 22, SEQ ID NO: 24, SEQ ID NO: 27, SEQ ID NO: 28, SEQ ID NO: 33, SEQ ID NO: 34, SEQ ID NO: 35, SEQ ID NO: 39, SEQ ID NO: 41, or an amino acid sequence at least 95% identical thereto, wherein the genome encodes a therapeutic protein, thereby treating the pancreatic disorder in the subject.

18. The method of claim 17, wherein the AAV vector transduces one or more pancreatic cell types with an efficiency that is about 5 times to about 200 times greater than that of AAV9.

19. The method of claim 17, wherein the AAV vector transduces one or more pancreatic cell types about 40 times to about 200 times greater than that of AAV9 delivered at the delivery point.

20. The method of claim 17, wherein the one or more pancreatic cell types comprise islet cells and the capsid protein comprises the amino acid sequence of SEQ ID NO: 1, SEQ ID NO: 7, SEQ ID NO: 10, SEQ ID NO: 12, SEQ ID NO: 16, SEQ ID NO: 20, SEQ ID NO:21, SEQ ID NO: 22, SEQ ID NO: 24, SEQ ID NO: 27, SEQ ID NO: 28, SEQ ID NO: 33, SEQ ID NO: 34, SEQ ID NO: 35, SEQ ID NO: 39, SEQ ID NO: 41, or an amino acid sequence at least 95% identical thereto.

21. The method of claim 17, wherein the one or more pancreatic cell types comprise duct cells and the capsid protein comprises the amino acid sequenced of SEQ ID NO: 1, SEQ ID NO: 2, SEQ ID NO: 7, SEQ ID NO: 8, SEQ ID NO: 10, SEQ ID NO: 12, SEQ ID NO: 13, SEQ ID NO: 15, SEQ ID NO: 16, SEQ ID NO: 17, SEQ ID NO: 20, SEQ ID NO: 21, SEQ ID NO: 24, SEQ ID NO: 25, SEQ ID NO: 28, SEQ ID NO: 33, SEQ ID NO: 35, SEQ ID NO: 39, and SEQ ID NO: 41, or a variant thereof exhibiting at least 95% identity thereto.

22. The method of claim 17, wherein the one or more pancreatic cell types comprise acinar cells and the capsid protein comprises the amino acid sequence of SEQ ID NO: 7, SEQ ID NO: 12, SEQ ID NO: 16, SEQ ID NO: 21, SEQ ID NO: 25, SEQ ID NO: 27, or an amino acid sequence at least 95% identical thereto.

23. The method of claim 17 wherein the AAV vector detargets a pancreatic cell type, resulting in transduction of the pancreatic cell type less than about 0.25 times that of AAV9.

24. The method of claim 17, wherein the AAV vector disseminates to liver, heart, and/or kidney, at a level less than about 0.1 times that of AAV9.

25. The method of claim 17, wherein the pancreatic disorder comprises type 1 diabetes, cystic fibrosis, hypercalcemia, or hypertriglyceridemia, type 2 diabetes, hereditary pancreatitis, and pancreatic cancer.
